SEQUENCE LISTING (8 sequences)

SEQ ID NO: 1 - DNA, 2444 bp, artificial sequence
  FLIP insert
gtcgacggatccataacttcgtataggataccttatacgaagttatctcaggtaccgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccctgtacaagaaacagaaaattgtggcaccagtgaaacagactttgaattttgaccttctcaagttggcgggagacgtcgagtccaaccctgggcccatgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcaccctcgggatacccgagcacacgtaccgctcccgcgtcaccctctccaaccagccctatatcaaggtccttaccctagccaacttcaccaccaaggatgagggcgactacttttgtgagcttcgagtctcgggcgcgaatcccatgagctccaataaaagtatcagtgtgtatagagacaaactggtcaagtgtggcggcataagcctgctggttcagaacacatcctggatgctgctgctgctgctttccctctccctcctccaagccctggacttcatttctctgtgatctagaagccataacttcgtatagtacacattatacgaagttatgtttaaacgcattagtcttccaattgaaaaaagtgatttaatttataccattttaattcagctttgtaaaaatgtatcaaagagatagcaaggtattcagttttagtaaacaagataattgctcctaaagtagccccttgaattctggttgctcgagccttctgttgggttaacctgaagaagtaatcccagcaagtgtttccaagatgtgcaggcaacgattctgtaaagtactgaagcctcattcaaacatagtatatgtgctgccgaagcgagcacttaacaaggcttgcggccgctacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtataacttcgtataaggtatcctatacgaagttatccattcaggctgtgctagcatcaatggcatggcacaaagcttagccataacttcgtataatgtgtactatacgaagttatcccgggttaac

SEQ ID NO: 2 - DNA, 103 bp, artificial sequence
  fflux RNAi
aaggtatattgctgttgacagtgagcgagctcccgtgaattggaatcctagtgaagccacagatgtaggattccaattcagcgggagcctgcctactgcctcg

SEQ ID NO: 3 - DNA, 2540 bp, artificial sequence
  pFLIP containing fflux RNAi
gtcgacggatccataacttcgtataggataccttatacgaagttatctcaggtaccgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccctgtacaagaaacagaaaattgtggcaccagtgaaacagactttgaattttgaccttctcaagttggcgggagacgtcgagtccaaccctgggcccatgaacccagccatcagcgtcgctctcctgctctcagtcttgcaggtgtccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcaccctcgggatacccgagcacacgtaccgctcccgcgtcaccctctccaaccagccctatatcaaggtccttaccctagccaacttcaccaccaaggatgagggcgactacttttgtgagcttcgagtctcgggcgcgaatcccatgagctccaataaaagtatcagtgtgtatagagacaaactggtcaagtgtggcggcataagcctgctggttcagaacacatcctggatgctgctgctgctgctttccctctccctcctccaagccctggacttcatttctctgtgatctagaagccataacttcgtatagtacacattatacgaagttatgtttaaacgcattagtcttccaattgaaaaaagtgatttaatttataccattttaattcagctttgtaaaaatgtatcaaagagatagcaaggtattcagttttagtaaacaagataattgctcctaaagtagccccttgaattccgaggcagtaggcaggctcccgctgaattggaatcctacatctgtggcttcactaggattccaattcacgggagctcgctcactgtcaacagcaatataccttctcgagccttctgttgggttaacctgaagaagtaatcccagcaagtgtttccaagatgtgcaggcaacgattctgtaaagtactgaagcctcattcaaacatagtatatgtgctgccgaagcgagcacttaacaaggcttgcggccgctacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtataacttcgtataaggtatcctatacgaagttatccattcaggctgtgctagcatcaatggcatggcacaaagcttagccataacttcgtataatgtgtactatacgaagttatcccgggttaac

SEQ ID NO: 4 - DNA, 7697 bp, modified lentivirus
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcctagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagatccataacttcgtataggataccttatacgaagttatctcaggtaccgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccctgtacaagaaacagaaaattgtggcaccagtgaaacagactttgaattttgaccttctcaagttggcgggagacgtcgagtccaaccctgggcccatgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcaccctcgggatacccgagcacacgtaccgctcccgcgtcaccctctccaaccagccctatatcaaggtccttaccctagccaacttcaccaccaaggatgagggcgactacttttgtgagcttcgagtctcgggcgcgaatcccatgagctccaataaaagtatcagtgtgtatagagacaaactggtcaagtgtggcggcataagcctgctggttcagaacacatcctggatgctgctgctgctgctttccctctccctcctccaagccctggacttcatttctctgtgatctagaagccataacttcgtatagtacacattatacgaagttatgtttaaacgcattagtcttccaattgaaaaaagtgatttaatttataccattttaattcagctttgtaaaaatgtatcaaagagatagcaaggtattcagttttagtaaacaagataattgctcctaaagtagccccttgaattccgaggcagtaggcaggctcccgctgaattggaatcctacatctgtggcttcactaggattccaattcacgggagctcgctcactgtcaacagcaatataccttctcgagccttctgttgggttaacctgaagaagtaatcccagcaagtgtttccaagatgtgcaggcaacgattctgtaaagtactgaagcctcattcaaacatagtatatgtgctgccgaagcgagcacttaacaaggcttgcggccgctacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtataacttcgtataaggtatcctatacgaagttatccattcaggctgtgctagcatcaatggcatggcacaaagcttagccataacttcgtataatgtgtactatacgaagttatcccgggttaaacgacctgcagccaagcttatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaa

SEQ ID NO: 5 - DNA, 103 bp, artificial sequence
  p53 RNAi
aaggtatattgctgttgacagtgagcgcccactacaagtacatgtgtaatgtgaagccacagatgtattacacatgtacttgtagtggatgcctactgcctcg

SEQ ID NO: 6 - DNA, 2530 bp, artificial sequence
  pFLIP containing p53 RNAi
tccataacttcgtataggataccttatacgaagttatctcaggtaccgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccctgtacaagaaacagaaaattgtggcaccagtgaaacagactttgaattttgaccttctcaagttggcgggagacgtcgagtccaaccctgggcccatgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcaccctcgggatacccgagcacacgtaccgctcccgcgtcaccctctccaaccagccctatatcaaggtccttaccctagccaacttcaccaccaaggatgagggcgactacttttgtgagcttcgagtctcgggcgcgaatcccatgagctccaataaaagtatcagtgtgtatagagacaaactggtcaagtgtggcggcataagcctgctggttcagaacacatcctggatgctgctgctgctgctttccctctccctcctccaagccctggacttcatttctctgtgatctagaagccataacttcgtatagtacacattatacgaagttatgtttaaacgcattagtcttccaattgaaaaaagtgatttaatttataccattttaattcagctttgtaaaaatgtatcaaagagatagcaaggtattcagttttagtaaacaagataattgctcctaaagtagccccttgaattccgaggcagtaggcatccactacaagtacatgtgtaatacatctgtggcttcactattacacatgtacttgtagtgggcgctcactgtcaacagcaatataccttctcgagccttctgttgggttaacctgaagaagtaatcccagcaagtgtttccaagatgtgcaggcaacgattctgtaaagtactgaagcctcattcaaacatagtatatgtgctgccgaagcgagcacttaacaaggcttgcggccgctacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtataacttcgtataaggtatcctatacgaagttatccattcaggctgtgctagcatcaatggcatggcacaaagcttagccataacttcgtataatgtgtactatacgaagttatcccgggtt

SEQ ID NO: 7 - DNA, 7698 bp, modified lentivirus
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcctagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagatccataacttcgtataggataccttatacgaagttatctcaggtaccgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccctgtacaagaaacagaaaattgtggcaccagtgaaacagactttgaattttgaccttctcaagttggcgggagacgtcgagtccaaccctgggcccatgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcaccctcgggatacccgagcacacgtaccgctcccgcgtcaccctctccaaccagccctatatcaaggtccttaccctagccaacttcaccaccaaggatgagggcgactacttttgtgagcttcgagtctcgggcgcgaatcccatgagctccaataaaagtatcagtgtgtatagagacaaactggtcaagtgtggcggcataagcctgctggttcagaacacatcctggatgctgctgctgctgctttccctctccctcctccaagccctggacttcatttctctgtgatctagaagccataacttcgtatagtacacattatacgaagttatgtttaaacgcattagtcttccaattgaaaaaagtgatttaatttataccattttaattcagctttgtaaaaatgtatcaaagagatagcaaggtattcagttttagtaaacaagataattgctcctaaagtagccccttgaattccgaggcagtaggcatccactacaagtacatgtgtaatacatctgtggcttcactattacacatgtacttgtagtgggcgctcactgtcaacagcaatataccttctcgagccttctgttgggttaacctgaagaagtaatcccagcaagtgtttccaagatgtgcaggcaacgattctgtaaagtactgaagcctcattcaaacatagtatatgtgctgccgaagcgagcacttaacaaggcttgcggccgctacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtataacttcgtataaggtatcctatacgaagttatccattcaggctgtgctagcatcaatggcatggcacaaagcttagccataacttcgtataatgtgtactatacgaagttatcccgggttaaacgacctgcagccaagcttatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaa

SEQ ID NO: 8 - DNA, 9207 bp, artificial sequence
  pFLIP containing puro2A-GFP
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaataaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagacgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtgccaccatggtggagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccaaacagaaaattgtggcaccagtgaaacagactttgaattttgaccttctcaagttggcgggagacgtcgagtccaaccctgggcccggcccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagcggccgcaagccttgttaagtgctcgcttcggcagcacatatactatgtttgaatgaggcttcagtactttacagaatcgttgcctgcacatcttggaaacacttgctgggattacttcttcaggttaacccaacagaaggctcgagcaaccagaattcaaggggctactttaggagcaattatcttgtttactaaaactgaataccttgctatctctttgatacatttttacaaagctgaattaaaatggtataaattaaatcacttttttcaattggaagactaatgcgtttaaacacgcggcgacgcgttcgaccgaataaaacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcggatctcgaccgcgtttgggcggtggctccctgccacgcggctccgaacagaagctgatctccgaagaggatctgacatgtgtttaaacctcgacttaattaagtcgagggtcgacggtatcgataagctcgcttcacgagatcatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

(19) United States
(12) Patent Application Publication
(10) Pub. No.: US 2009/0217397 A1
(43) Pub. Date: Aug. 27, 2009

(54) Cre-lox based gene knockdown constructs and methods of use thereof

(76) Inventors: Patrick Stern, Cambridge, MA (US); Richard Hynes, Winchester, MA (US)

Correspondence Address: Pearl Cohen Zedek Latzer, LLP, 1500 Broadway, 12th Floor, New York, NY, 10036, US

(21) Appl. No.: 11/808,553

(22) Filed: Jun. 11, 2007

Related U.S. Application Data
(60) Provisional application No. 60/812,608, filed on Jun. 12, 2006.

(51) Int. Cl. C12N 15/09; C12N 15/63; A01K 67/02; A01K 67/027; C12N 5/10

(52) U.S. Cl. 800/13; 435/320.1; 800/25; 435/455; 435/325

(57) ABSTRACT

The present invention relates to vectors, compositions and methods for conditional, Cre-lox regulated, RNA interference. The vectors allow for spatial and temporal control of miRNA expression in vivo.

### CROSS-REFERENCE TO RELATED APPLICATIONS

This Application claims the benefit of U.S. Provisional Application Ser. No. 60/812,608, filed Jun. 12, 2006, which is hereby incorporated herein in its entirety.

### FIELD OF THE INVENTION

The present invention relates to vectors and their use in a Cre-lox based method for conditional RNA interference.

### BACKGROUND OF THE INVENTION

RNA interference (RNAi) is an extremely verstatile tool for inhibition of gene expression. RNAi is based on the introduction of double stranded RNA (dsRNA) molecules into cells, whereby one strand is complementary to the coding region of a target gene. Through pairing of the specific mRNA with the introduced RNA molecule, the mRNA is degraded by a cellular mechanism. Short (30 bp) interfering RNA duplexes (siRNA) have been shown to be effective, and do not provoke an immune response, extending the application to mammalian cells. Small hairpin RNAs (shRNAs) transcribed in vivo, are able to trigger degradation of corresponding mRNAs similar to the siRNAs. Micro RNAs (miRNAs) are the endogenous form of shRNAs that carry out the gene silencing function in vivo.

shRNA expression has been accomplished using gene expression vectors, with RNA polymerase III (Pol III) or Polymerase II (Po III) promoters, with expression occurring in mice injected with the shRNA expression vectors, however, gene inhibition was temporally and spatially restricted. Moreover stable integration of the construct is not readily accomplished or validated in current systems.

### BRIEF DESCRIPTION OF THE DRAWINGS

FIG. 1 depicts one embodiment of the organization and expression of constructs of this invention. A. Shematic representation of an embodiment of the pFLIP inserts of this invention. Two pairs of mutated loxP sites and their orientation are depicted, as well as the positioning of the positioning of the selectable marker sequences and miRNA sequence, with respect thereto. Schematic shows elements prior to and following Cre-mediated recombination.

FIG. 2 shows the results of a FACS analysis of pFLIP expression in “uninduced” and “induced” states in Lewis Lung carcinoma cell lines (LL2) infected with an MSCV retrovirus expressing pFLIP. The panels show FACS analysis for expressed markers.

FIG. 3 demonstrates expression and knockdown ability of an embodiment of the retroviral pFLIP vectors of this invention in the presence of Cre. Primary mouse embryo fibroblasts (MEFs) were infected with MSCV retrovirus expressing pFLIP comprising an RNAi against the tumor suppressor p53. Cells were selected with puromycin and infected with retrovirus expressing Cre recombinase. Cells were treated for 5 hours with doxorubicin, which causes DNA damage and induces p53 expression.

### DETAILED DESCRIPTION OF THE INVENTION

The present invention provides, in one embodiment, constructs and methods for conditionally reducing expression of a coding sequence in a cell or animal, comprising contacting the cell with a vector comprising a first selectable marker in sense orientation, and a second selectable marker fused in frame to an miRNA sequence, in antisense orientation, whereby the marker sequences are flanked by two pairs of loxP sites, which sites are initially inverted in orientation, in cells capable of expressing a Cre recombinase.

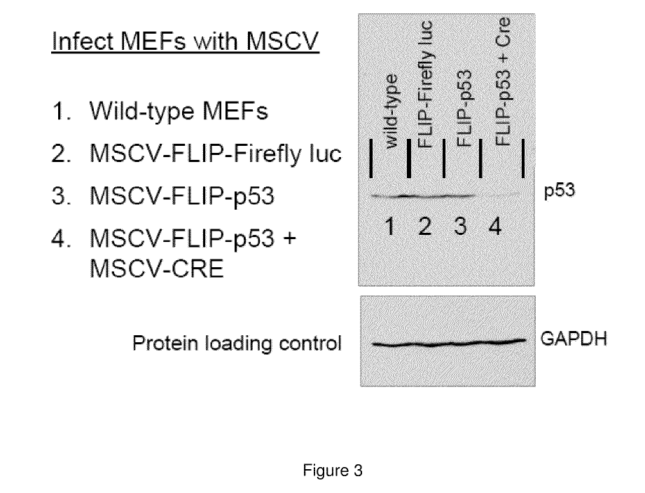

Conditionally reduced expression of a coding sequence was demonstrated herein, with the use of a retroviral vector pFLIP, which comprises, in some embodiments, a first pair of loxP sequences, inverted in orientation, with respect to each other, a first nucleic acid encoding a first selectable marker in sense orientation, wherein said nucleic acid is positioned between said first pair of loxP sequences, a second nucleic acid encoding a second selectable marker, fused in frame to an miRNA sequence of interest in antisense orientation, said second nucleic acid is positioned between said first pair of loxP sequences, and said second nucleic acid is 3′ with regard to said first nucleic acid, a second pair of loxP sequences, inverted in orientation, with respect to each other, wherein said first loxP sequenced of said second pair is positioned between said first and said second nucleic acid, and said second loxP sequence in said second pair is positioned 3′ with respect to said first pair of loxP sequences, and said second pair of loxP sequences differs from that of said first pair of loxP sequences. Cre expression in MEF cells infected with pFLIP expressed the shRNA, and demonstrated reduced p53 protein levels (FIG. 3).

In one embodiment, this invention provides a method of conditionally reducing expression of a coding sequence in a target cell, said method comprising contacting said target cell with a vector comprising:
i. a first pair of loxP sequences, inverted in orientation, with respect to each other;
ii. a first nucleic acid encoding a first selectable marker in sense orientation, wherein said nucleic acid is positioned between said first pair of loxP sequences;
iii. a second nucleic acid encoding a second selectable marker, fused in frame to an miRNA sequence of interest in antisense orientation, said second nucleic acid is positioned between said first pair of loxP sequences, and said second nucleic acid is 3′ with regard to said first nucleic acid;
iv. a second pair of loxP sequences, inverted in orientation, with respect to each other, wherein said first loxP sequenced of said second pair is positioned between said first and said second nucleic acid, and said second loxP sequence in said second pair is positioned 3′ with respect to said first pair of loxP sequences, and said second pair of loxP sequences differs from that of said first pair of loxP sequences;
   wherein said target cell is capable of expressing a Cre recombinase and whereby, following Cre-mediated recombination, said miRNA agent is expressed and reduces expression of said coding sequence, thereby conditionally reducing expression of a coding sequence in a target cell.

In one embodiment, the term “vector” refers to a nucleic acid molecule capable of transporting another nucleic acid to which it has been linked. In one embodiment, the vector is a genomic integrated vector, or “integrated vector”, which can become integrated into the chromsomal DNA of the host cell. In another embodiment, the vector is an episomal vector, i.e., a nucleic acid capable of extra-chromosomal replication in an appropriate host, such as, for example a eukaryotic host cell. The vector according to this aspect of the present invention may be, in other embodiments, a plasmid, a bacmid, a phagemid, a cosmid, a phage, a virus or an artificial chromosome.

A nucleic acid of the present invention will generally contain phosphodiester bonds in one embodiment, or in another embodiment, nucleic acid analogs are included, that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate, phosphorodithioate, O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). These modifications of the ribose-phosphate backbone or bases may be done to facilitate the addition of other moieties such as chemical constituents, including 2′O-methyl and 5′ modified substituents, or to increase the stability and half-life of such molecules in physiological environments.

The nucleic acids may be single stranded or double stranded, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine and hypoxathanine, etc. Thus, for example, chimeric DNA-RNA molecules may be used such as described in Cole-Strauss et al., Science 273:1386 (1996) and Yoon et al., PNAS USA 93:2071 (1996).

The vectors of this invention comprise, inter alia, an miRNA agent specific for a coding sequence.

The term “miRNA agent” refers, in one embodiment, to an agent that modulates expression of a target gene by an RNA interference mechanism. Micro-RNAs are a very large group of small RNAs produced naturally in organisms, which in one embodiment, regulates the expression of target genes. Founding members of the micro-RNA family are let-7 and lin-4. The let-7 gene encodes a small, highly conserved RNA species that regulates the expression of endogenous protein-coding genes during worm development. The active RNA species is transcribed initially as an ^{˜}70 nt precursor, which is post-transcriptionally processed into a mature ^{˜}21 nt form. Both let-7 and lin-4 are transcribed as hairpin RNA precursors, which are processed to their mature forms by Dicer enzyme.

In one embodiment the miRNA agent comprises double-stranded RNA, which can form a hairpin structure. The miRNA agents employed, in another embodiment, are small ribonucleic acid molecules, or oligoribonucleotides, that are present in duplex structures, such as, in one embodiment, two distinct oligoribonucleotides hybridized to each other, or in another embodiment, a single ribooligonucleotide that assumes a hairpin structure to produce a duplex structure.

In one embodiment, miRNA agent does not exceed about 100 nt in length, and typically does not exceed about 75 nt length, where the length in certain embodiments is less than about 70 nt. In one embodiment, the miRNA agent of this invention has a length about 15 to 40 bp, or in another embodiment, about 20 and 29 bps, or in another embodiment, 25 and 35 bps, or in another embodiment, about 20 and 35 bps, or in another embodiment, about 20 and 40 bps, or in another embodiment, 21 bp, or in another embodiment, 22 bp.

In one embodiment, the nucleic acids/oligonucleotides comprising the miRNA agent may be synthesized on an Applied Bio Systems oligonucleotide synthesizer according to specifications provided by the manufacturer. In another embodiment, the nucleic acids/oligonucleotides or modified oligonucleotides may be synthesized by any number of means as is generally known in the art, and as is described hereinbelow.

In one embodiment, the miRNA agent encodes an interfering ribonucleic acid. In one embodiment, the miRNA agent is a transcriptional template of the interfering ribonucleic acid. According to this aspect of the invention, and in one embodiment, the transcriptional template is typically a DNA that encodes the interfering ribonucleic acid. The DNA may be present in a vector, such as, and in one embodiment, a plasmid vector, or in another embodiment, a viral vector, or any other vector, as will be known to one skilled in the art.

In one embodiment, the term “coding sequence” refers to a nucleic acid sequence that “encodes” a particular polypeptide or peptide. In one embodiment, the coding sequence is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from procaryotic or eukaryotic mRNA, genomic DNA sequences from procaryotic or eukaryotic DNA, and even synthetic DNA sequences. A transcription termination sequence will usually be located 3′ to the coding sequence.

In one embodiment the term “coding sequence”, includes DNA sequences that encode a polypeptide, as well as DNA sequences that are transcribed into inhibitory antisense molecules.

In one embodiment, the term “reducing expression”, as it refers to vectors and their use according to the methods of this invention, refers to a diminishment in the level of expression of a gene when compared to the level in the absence of the miRNA agent.

In one embodiment, reduced expression may be affected at the transcriptional or translational level, or a combination thereof.

According to this aspect of the invention, reduced expression using the vectors, and/or according to the methods of this invention, is specific. In one embodiment, the reduction in expression is via an ability to inhibit a target gene without manifest effects on other genes of the cell. The consequences of inhibition can be confirmed, in other embodiments, by examination of the outward properties of the cell or organism or by biochemical techniques such as RNA solution hybridization, nuclease protection, Northern hybridization, gene expression monitoring with a microarray, antibody binding, enzyme linked immunosorbent assay (ELISA), Western blotting, radioimmunoassay (RIA), other immunoassays, and fluorescence activated cell analysis (FACS).

In one embodiment, the miRNA agent is an shRNA, which specifically inactivates p53, as exemplified hereinbelow.

In one embodiment, the vectors and methods of utilizing the same for reducing expression of a target gene may result in inhibition of target gene expression of greater than 10%, 33%, 50%, 75%, 80%, 85%, 90%, 95% or 99% as compared to a cell not subjected to the vectors and methods of utilizing the same for reducing expression. In another embodiment, lower doses of administered miRNA agent, and longer times following administration may result in inhibition in a smaller fraction of cells (e.g., at least 10%, 20%, 50%, 75%, 90%, or 95% of targeted cells).

In one embodiment, this invention provides for a method of conditionally reduced expression of a coding sequence in a target cell. In one embodiment, the term “conditionally reduced expression” refers to the flexibility inherent in the methods/vectors of this invention, which enable regulation of reducing expression of a coding sequence in a target cell. In one embodiment, reducing expression via the vectors/methods of this invention is controlled over time, or in a cell or tissue-specific manner, such that production of the miRNA agent is not constant.

Expression of the miRNA agent within a target cell, in one embodiment of this invention, takes advantage of a lox/cre system. In one embodiment, miRNA agent expression is dependent upon the presence of a Cre recombinase. In one embodiment, abrogation of miRNA expression is dependent upon the presence of a Cre recombinase.

In one embodiment, the cre recombinase, is derived from a P1 bacteriophage (Abremski and Hoess, J. Biol. Chem. 259(3):1509-1514 (1984)) which acts on a specific 34 base pair DNA sequence known as “loxP” (locus of crossover), which is, in turn, comprised of two 13 base pair inverted repeats (serving as the recombinase binding sites) flanking an 8 base pair core sequence (Current Opinion in Biotechnology 5:521-527 (1994). Cre catalyzes the rearrangement of DNA sequences that contain loxP sites. Recombination between two loxP sites (catalyzed by the cre protein) causes, in certain cases, the loss of sequences flanked by these sites [for a review see N. Kilby et al, Trends Genet., 9:413-421 (1993)].

In some embodiments, this invention utilizes two sets of loxP sites, whose sequences differ. In one embodiment, one pair of the loxP sites may be WT, while the other may be mutated, or in another embodiment, both are mutated. In one embodiment, the loxP sets are oriented initially with inverted orientation, such that regions of the vector undergo inversion, following exposure to a Cre-recombinase. Following such inversion, one of the pairs of loxP sites are co-aligned, thus in the presence of a Cre-recombinase, excision can occur.

In one embodiment, the two pairs of loxP sites are chosen so as to minimize recombination therebetween, as exemplified herein.

Cre works in simple buffers, such as, in one embodiment, with magnesium or, in another embodiment, spermidine as a cofactor, as is well known in the art. The DNA substrates acted on by cre may be, in one embodiment, in linear, or, in another embodiment, in a supercoiled configuration.

In one embodiment, the cre sequence is as that described in N. Sternberg et al, J. Mol. Biol., 187:197-212 (1986). In another embodiment, the cre recombinase may be obtained from commercial sources (for example from Novagen, Catalog No. 69247-1).

In one embodiment, cre recombinase will be expressed in a target cell of this invention. In another embodiment, the target cell will be engineered to express cre by any means as will be known to one skilled in the art.

In one embodiment, the terms “homology”, “homologue” or “homologous”, refer to a, which exhibits, in one embodiment at least 70% correspondence with the indicated sequence. In another embodiment, the sequence exhibits at least 72% correspondence with the indicated sequence. In another embodiment, the sequence exhibits at least 75% correspondence with the indicated sequence. In another embodiment, the sequence exhibits at least 80% correspondence with the indicated sequence. In another embodiment, the sequence exhibits at least 82% correspondence with the indicated sequence. In another embodiment, the sequence exhibits at least 85% correspondence with the indicated sequence. In another embodiment, the sequence exhibits at least 87% correspondence with the indicated sequence. In another embodiment, the sequence exhibits at least 90% correspondence with the indicated sequence. In another embodiment, the sequence exhibits at least 92% correspondence with the indicated sequence. In another embodiment, the sequence exhibits at least 95% or more correspondence with the indicated sequence. In another embodiment, the sequence exhibits at least 97% correspondence with the indicated sequence. In another embodiment, the sequence exhibits at least 99% correspondence with the indicated sequence. In another embodiment, the sequence exhibits 95%-100% correspondence with the indicated sequence. Similarly, as used herein, the reference to a correspondence to a particular sequence includes both direct correspondence, as well as homology to that sequence as herein defined.

Homology, as used herein, may refer to sequence identity, or may refer to structural identity, or functional identity. By using the term “homology” and other like forms, it is to be understood that any molecule, that functions similarly, and/or contains sequence identity, and/or is conserved structurally so that it approximates the reference sequence, is to be considered as part of this invention.

Homology may be determined in the latter case by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

An additional means of determining homology is via determination of candidate sequence hybridization, methods of which are well described in the art (See, for example, Nucleic Acid Hybridization, Hames and Higgins, Eds. (1985); Molecular Cloning, Sambrook and Russell, eds. (2001), and Current Protocols in Molecular Biology, Ausubel et al. eds, 1989). For example, methods of hybridization may be, in one embodiment, carried out under moderate to stringent conditions, to the complement of a DNA encoding a native peptide or protein of interest. Hybridization conditions may be, for example, overnight incubation at 42° C. in a solution comprising: 10-20% formamide, 5×SSC (150 millimolar (mM) NaCl, 15 mM trisodium citrate), 50 mM sodium phosphate (pH 7.6), 5×Denhardt's solution, 10% dextran sulfate, and 20 micrograms (μg)/milliliter (ml) denatured, sheared salmon sperm DNA. Each method represents a separate embodiment of the present invention.

In another embodiment, mutated loxP sites, may be employed in the vectors and/or methods of this invention.

In one embodiment, the constructs of this invention will comprise a promoter, operatively linked to the first nucleic acid sequence encoding a selection marker. In one embodiment, the term “promoter” refers to a nucleic acid sequence, which regulates expression of a nucleic acid, operably linked thereto. Such promoters are known to be cis-acting sequence elements required for transcription as they serve to bind DNA dependent RNA polymerase, which transcribes sequences present downstream thereof.

The term “operably linked”, in one embodiment, refers to a relationship permitting the sequences to function in their intended manner. A vector comprising a regulatory sequence “operably linked” to a coding sequence is ligated in such a way that expression of the nucleic acid sequence is achieved under conditions compatible with the control sequences.

In one embodiment, the promoter will be an RNA polymerase III promoter.

In one embodiment, a promoter, including an engineered promoter used in the vectors and methods of this invention, may be one known to confer cell-type specific expression of a sequence operatively linked to thereto. For example, and in one embodiment, a promoter specific for myoblast gene expression can be operatively linked to an miRNA for a coding sequence of interest, a reporter gene, or a coding sequence of interest, to confer muscle-specific expression thereof. Muscle-specific regulatory elements which are known in the art include upstream regions from the dystrophin gene (Klamut et al., (1989) Mol. Cell Biol. 9:2396), the creatine kinase gene (Buskin and Hauschka, (1989) Mol. Cell Biol. 9:2627) and the troponin gene (Mar and Ordahl, (1988) Proc. Natl. Acad. Sci. USA. 85:6404).

In another embodiment, promoters used in the vectors and methods of this invention, specific for other cell types known in the art (e.g., the albumin enhancer for liver-specific expression; insulin regulatory elements for pancreatic islet cell-specific expression; various neural cell-specific regulatory elements, including neural dystrophin, neural enolase and A4 amyloid promoters) may be used, and represent an embodiment of this invention. In another embodiment, a promoter or regulatory element, which can direct constitutive expression of a sequence operatively linked thereto, in a variety of different cell types, such as a viral regulatory element, may be used. Examples of viral promoters commonly used to drive gene expression include those derived from polyoma virus, Adenovirus 2, cytomegalovirus and Simian Virus 40, and retroviral LTRs.

In another embodiment, a regulatory element, which provides inducible expression of a gene linked thereto, may be used. The use of an inducible promoter may allow, in another embodiment, for an additional means of modulating the product of the coding sequence in the cell. Examples of potentially useful inducible regulatory systems for use in eukaryotic cells include hormone-regulated elements (e.g., see Mader, S. and White, J. H. (1993) Proc. Natl. Acad. Sci. USA 90:5603-5607), synthetic ligand-regulated elements (see, e.g., Spencer, D. M. et al 1993) Science 262:1019-1024) and ionizing radiation-regulated elements (e.g., see Manome, Y. Et al. (1993) Biochemistry 32:10607-10613; Datta, R. et al. (1992) Proc. Natl. Acad. Sci. USA 89: 1014-10153). Additional tissue-specific or inducible regulatory systems may be developed for use in accordance with the invention.

In one embodiment, the term “capable of expressing a Cre recombinase” refers to a cell that endogenously expresses the Cre recombinase, or in another embodiment, is engineered to express a Cre recombinase.

In one embodiment, the cell is in a culture system, or in another embodiment, in a body of a subject, or in another embodiment, is ex-vivo cultured, and following transfection or tranduction with a vector of this invention, is reintroduced to the subject from which the cell was taken. In one embodiment, the cell is a stem or progenitor cell. In another embodiment, the cell is a mature, differentiated cell. In one embodiment, the cell is a human cell in origin, or in another embodiment, the cell is murine in origin.

In one embodiment, the terms “Cells,” “host cells” or “target cells” are used interchangeably, and refer, in one embodiment, not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

In another embodiment, the cell is a diseased cell. In one embodiment, the cell is infected, or in another embodiment, the cell is transformed or neoplastic. In another embodiment, the cell is obtained from a subject with a disease whose etiology is associated with a genetic mutation. In another embodiment, the cell is obtained from a subject with a disease, where an inappropriate immune or inflammatory response has been initiated.

In one embodiment, the target cell of any method of the present invention may be a cancer cell or neoplastic cell. “Neoplastic cell” refers, in one embodiment, to a cell whose normal growth control mechanisms are disrupted (typically by accumulated genetic mutations), thereby providing potential for uncontrolled proliferation. Thus, “neoplastic cell” can include, in one embodiment, both dividing and non-dividing cells. In one embodiment, neoplastic cells may include cells of tumors, neoplasms, carcinomas, sarcomas, leukemias, lymphomas, and others. In another embodiment, “neoplastic cells” may include central nervous system tumors, such as, for example brain tumors. These may include, in other embodiments, glioblastomas, astrocytomas, oligodendrogliomas, meningiomas, neurofibromas, ependymomas, schwannomas or neurofibrosarcomas. In another embodiment, “neoplastic cells” can include either benign or malignant neoplastic cells. In another embodiment, “neoplastic cells” can include any other type of cancer known in the art.

In one embodiment, the target cell may be an infected cell. In another embodiment, the target cell may be a pathogenic cell. In another embodiment, the target cell may mediate autoimmunity or another disease state. In another embodiment, the target cell may comprise a mutated cellular gene necessary for a physiological function. In one embodiment, the mutated product results in disease in the subject. According to this aspect of the invention, the vectors/methods of this invention may be employed to silence a defective gene, and may further be followed by delivery of a wild-type copy of the desired gene.

It is to be understood that any cell comprising a vector of this invention, or utilized for the methods of this invention, is to be considered as part of this invention, and represents an embodiment thereof.

According to this aspect of the invention, and in one embodiment, following Cre-mediated recombination in the target cell, the miRNA agent is expressed and reduces expression of the coding sequence, thereby conditionally reducing expression of a coding sequence in the target cell.

In another embodiment, the vector is a retroviral vector. In one embodiment, the retroviral vector of this invention may correspond to one as exemplified herein.

A retroviral or retrovirus vector, as used herein, is a vector, which comprises at least one component part derivable from a retrovirus. In one embodiment, the component part is involved in the biological mechanisms by which the vector infects cells, expresses genes or is replicated. The term “derivable”, in one embodiment, refers to the fact that the sequence need not necessarily be obtained from a retrovirus but instead could be derived therefrom. By way of example, the sequence may be prepared synthetically or by use of recombinant DNA techniques.

The retroviral vectors of this invention may be derived from any member of the family of retroviridae.

In one embodiment, the retroviral vectors of this invention comprise sufficient retroviral genetic information to allow packaging of an RNA genome, in the presence of packaging components, into a viral particle capable of infecting a target cell. In one embodiment, infection of the target cell includes reverse transcription and integration into the target cell genome. The retroviral vectors of this invention may carry, in one embodiment, non-viral coding sequences which are to be delivered by the vector to the target cell. In one embodiment, the retroviral vectors of this invention are incapable of independent replication to produce infectious retroviral particles within the final target cell. In one embodiment, the retroviral vectors of this invention will lack a functional gag-pol and/or env gene and/or other genes essential for replication.

In one embodiment, the vectors and methods of this invention may employ the use of enhancer sequences. In one embodiment, the term “enhancer” refers to a DNA sequence, which binds to other protein components of the transcription initiation complex and may thus facilitate the initiation of transcription directed by its associated promoter.

In another embodiment, the vectors and their use according to the present invention include at least two selectable markers, which may serve to indicate inversion and excision mediated by a Cre-recombinase, as described herein. In one embodiment, the selectable marker comprises an antibiotic resistance cassette, by means well known to one skilled in the art. In one embodiment, the resistance cassette is for conferring resistance to ampicillin, bleomycin, chloramphenicol, gentamycin, hygromycin, kanamycin, lincomycin, methotrexate, phosphinothricin, puromycin, or tetracycline, or derivatives thereof.

In another embodiment, the selectable marker may comprise nucleic acid sequences encoding for a reporter protein, such as, for example, green fluorescent protein (GFP), DS-Red (red fluorescent protein), acetohydroxyacid synthase (AHAS), beta glucoronidase (GUS), secreted alkaline phosphatase (SEAP), beta-galactosidase, chloramphenicol acetyltransferase (CAT), horseradish peroxidase (HRP), luciferase, nopaline synthase (NOS), octopine synthase (OCS), or derivatives thereof, or any number of other reporter proteins known to one skilled in the art.

In another embodiment, the vector may further include an origin of replication, and may be a shuttle vector, which can propagate both in bacteria, such as, for example, E. Coli (wherein the vector comprises an appropriate selectable marker and origin of replication) and be compatible for propagation in vertebrate cells, or integration in the genome of an organism of choice.

The nucleic acids may be introduced into tissues or host cells by any number of routes, including viral infection, microinjection, or fusion of vesicles. Jet injection may also be used for intra-muscular administration, as described by Furth et al. (1992), Anal Biochem 205:365-368. The nucleic acids may be coated onto gold microparticles, and delivered intradermally by a particle bombardment device, or “gene gun” as described in the literature (see, for example, Tang et al. (1992), Nature 356:152-154), where gold microprojectiles are coated with the DNA, then bombarded into skin cells. Expression vectors may be used to introduce the nucleic acids into a cell.

In one embodiment, the vectors of this invention may be fed directly to, injected into, the host organism containing the target gene. The vectors of this invention may be directly introduced into the cell (i.e., intracellularly); or introduced extracellularly into a cavity, interstitial space, into the circulation of an organism, introduced orally, etc. Methods for oral introduction include direct mixing of the vector with food of the organism. Physical methods of introducing the vectors include injection directly into the cell or extracellular injection into the organism of a solution comprising the vector. The vectors may be introduced in an amount, which allows delivery of at least one copy per cell. Higher doses (e.g., at least 5, 10, 100, 500 or 1000 copies per cell) of the vectors may yield more effective inhibition; lower doses may also be useful for specific applications.

In other embodiments, a hydrodynamic administration protocol is employed, and may be as described in Chang et al., J. Virol. (2001) 75:3469-3473; Liu et al., Gene Ther. (1999) 6:1258-1266; Wolff et al., Science (1990) 247: 1465-1468; Zhang et al., Hum. Gene Ther. (1999) 10:1735-1737: and Zhang et al., Gene Ther. (1999) 7:1344-1349, each of which represents an embodiment of this invention.

In other embodiments, delivery protocols of interest may include, but are not limited to: those described in U.S. Pat. Nos. 5,985,847, or 5,922,687, WO/11092; Acsadi et al., New Biol. (1991) 3:71-81; Hickman et al., Hum. Gen. Ther. (1994) 5:1477-1483; or Wolff et al., Science (1990) 247: 1465-1468, and others, as will be appreciated by one skilled in the art.

The methods of this invention comprise the step of contacting a target cell with a vector of this invention. In one embodiment, the terms “contacting”, “contact” or “contacted” indicate, direct or, in another embodiment, indirect exposure of the cell to a vector, compound or composition comprising the vectors of this invention. It is envisaged that, in another embodiment, indirect supply to the cell may be via provision in a culture medium that surrounds the cell, or via parenteral administration in a body of a subject, whereby the vector ultimately contacts a cell via peripheral circulation (for further detail see, for example, Methods in Enzymology Vol. 1-317, Rubin and Dennis, eds, (1955-2003) and Current Protocols in Molecular Biology, Ausubel, et al, eds (1998), Molecular Cloning: A Laboratory Manual, Sambrook and Russell, eds., (2001), or other standard laboratory manuals). It is to be understood that any direct means or indirect means of intracellular access of a vector, or composition comprising the same of this invention represents an embodiment thereof.

In one embodiment, the target cell is contacted with a vector/composition comprising the same, of this invention, in vivo, in vitro or ex-vivo. In one embodiment, cells may be procured from a subject, contacted with a vector of this invention, and reintroduced into the subject. In one embodiment, the cell is a stem or progenitor cell, and reintroduction into the subject may be followed, in another embodiment, by stimulation of differentiation of the contacted cell, in vivo.

In another embodiment, Cre recombinase is expressed at specific times during development.

In another embodiment, this invention provides for the generation of a non-human animal with reduced expression of a coding sequence, wherein the reduced expression is produced according to the methods, and/or utilizing the vectors of this invention.

Transgenic mice, may, in one embodiment, be derived using the vectors/methods of this invention, according to Hogan, et al., “Manipulating the Mouse Embryo: A Laboratory Manual”, Cold Spring Harbor Laboratory (1988) which is incorporated herein by reference. Embryonic stem cells may, in another embodiment, be manipulated according to published procedures (Teratocarcinomas and embryonic stem cells: a practical approach, E. J. Robertson, ed., IRL Press, Washington, D.C., 1987; Zjilstra et al., Nature 342:435-438 (1989); and Schwartzberg et al., Science 246:799-803 (1989), each of which is incorporated herein by reference). Zygotes may be manipulated, in another embodiment, according to known procedures; for example see U.S. Pat. No. 4,873,191, Brinster et al., PNAS 86:7007 (1989); Susulic et al., J. Biol. Chem. 49:29483 (1995), and Cavard et al., Nucleic Acids Res. 16:2099 (1988), hereby incorporated by reference. Tetraploid blastocyst complementation may also be utilized to achieve non-human animals, which express the vectors of this invention, according to methods as exemplified herein, or, as are well known in the art.

In one embodiment, this invention provides a method of producing an animal genetically inactivated for a coding sequence, the method comprising contacting an embryonic stem cell with a vector of this invention which may be used for gene silencing, injecting the contacted embryonic stem cell to a blastocyst of an animal and obtaining an animal expressing the vector, whereby, following Cre-mediated recombination in the animal, the miRNA agent is expressed and reduces expression of the coding sequence, thereby being a method of producing an animal genetically inactivated for a coding sequence.

In another embodiment, the method of conditionally reducing expression of a coding sequence, as described and exemplified herein, may be therapeutic. In one embodiment, the term “therapeutic” refers to the fact that when in contact with a cell in a subject in need, provides a beneficial effect.

In one embodiment, the compositions/vectors and methods of conditionally reducing expression of a coding sequence of this invention prevent inappropriate expression of an encoded protein in a subject. Some examples include endogenous proteins which are mutated, and produces a non-functional protein, or an over-expressed protein, which in another embodiment, may be non-functional, or in another embodiment, pathogenic.

In one embodiment, the encoded protein may include cytokines, such as interferons or interleukins, or their receptors. According to this aspect of the invention, and in one embodiment, inappropriate expression patterns of cytokines may be altered to produce a beneficial effect, such as for example, a biasing of the immune response toward a Th1 type expression pattern, or a Th2 pattern in infection, or in autoimmune disease, wherein altered expression patterns may prove beneficial to the host. In these cases, and in one embodiment, conditionally reducing expression of the inappropriate or non-protective cytokine/receptor may be followed by delivery of an appropriate cytokine, or a vector/nucleic acid for expressing the same.

In another embodiment, the encoded protein may include an enzyme, such as one involved in glycogen storage or breakdown. In another embodiment, the encoded protein may include a transporter, such as an ion transporter, for example CFTR, or a glucose transporter, or other transporters whose inappropriate expression results in a variety of diseases. As described hereinabove, and in another embodiment, conditionally reducing expression of the encoded proteins, according to this aspect of the invention, may be followed by delivery of a wild-type protein, or a plasmid encoding same, or a mutated protein, which results in a therapeutic effect in the subject.

In another embodiment, the encoded protein may include a receptor, such as one involved in signal transduction within a cell. Some examples include as above, cytokine receptors, leptin receptors, transferring receptors, etc., or any receptor wherein altered expression results in inappropriate or inadequate signal transduction in a cell.

It is to be understood that any encoded protein, wherein conditionally reducing expression of the product is therapeutic to a subject is to be considered as part of this invention, and methods/vectors to provide wild-type or otherwise therapeutic versions of the encoded protein to the subject, following conditional reduction of expression of the mutated version, is to be considered as part of this invention, and embodiments thereof.

In another embodiment, the vectors/methods of this invention may be utilized to conditionally reduce expression of an oncogene, whose expression promotes cancer-related events. In one embodiment, the conditionally reduced expression of oncogenes comprising ABLI, BCLI, BCL2, BCL6, CBFA2, CBL, CSFIR, ERBA, ERBB, EBRB2, ETSI, ETS1, ETV6, FOR, FOS, FYN, HCR, HRAS, JUN, KRAS, LCK, LYN, MDM2, MLL, MYB, MYC, MYCLI, MYCN, NRAS, PIM 1, PML, RET, SRC, TALI, TCL3, YES, or combinations thereof, may be effected via the vectors/compositions/methods of this invention. In another embodiment, vectors/methods of this invention may be utilized to conditionally reduce expression of a Prostate Tumor Inducing Gene, which may comprise in one embodiment, PTI-1, PTI-2, PTI-3 or combinations thereof.

In one embodiment, the vectors/methods of this invention may be utilized to conditionally reduce expression of genes whose products promote angiogenesis, such as, for example, and in one embodiment, VEGF, VEGF receptor, erythropoietin, or combinations thereof. In another embodiment, the coding sequence for which conditional reducing expression is desired may comprise a matrix metalloproteinase, wherein reduction of expression prevents, in one embodiment, metastasis of cancerous cells, or, in another embodiment, tissue necrosis in infectious or inflammatory diseases.

In another embodiment, the vectors/compositions/methods of this invention may be utilized to conditionally reduce expression of a mutated rhodopsin gene. Autosomal dominant retinitis pigmentosa (ADRP) is characterized by the substitution of histidine for proline at codon 23 (P23H) in their rhodopsin gene, resulting in photoreceptor cell death from the synthesis of the abnormal gene product. In one embodiment, P23H mutant mRNAs may be targeted for conditional reduction of expression.

In another embodiment, the vectors/compositions/methods of this invention may be utilized to reverse effects of high glucose on progression of diabetic retinopathy. High glucose environments can result in chronically increased nitric oxide (NO) activity, which leads to endothelial cell dysfunction and impaired blood retinal barrier integrity characteristic of diabetic retinopathy.

In one embodiment, NOS synthesis may be conditionally reduced, in a tissue specific manner, in another embodiment, via the use of miRNAs targeted against VEGF, iNOS, or eNOS using the vectors/compositions and methods, as described hereinabove. In another embodiment, glucose transporters may be similarly targeted for therapeutic purposes in diabetic retinopathy.

In another embodiment, the vectors/compositions and methods for reducing expression of a coding sequence may be applied in a subject with a disease, where the disease may comprise, but is not limited to: muscular dystrophy, cancer, cardiovascular disease, hypertension, infection, renal disease, neurodegenerative disease, such as alzheimer's disease, parkinson's disease, huntington's chorea, Creuztfeld-Jacob disease, autoimmune disease, such as lupus, rheumatoid arthritis, endocarditis, Graves' disease or ALD, respiratory disease such as asthma or cystic fibrosis, bone disease, such as osteoporosis, joint disease, liver disease, disease of the skin, such as psoriasis or eczema, ophthalmic disease, otolaryngeal disease, other neurological disease such as Turret syndrome, schizophrenia, depression, autism, or stoke, or metabolic disease such as a glycogen storage disease or diabetes. It is to be understood that any disease whereby reduced expression of a particular protein, which can be accomplished via the use of the vectors or cells or compositions, or via the methods of this invention, is to be considered as part of this invention.

In another embodiment, this invention provides a method of conditionally expressing a coding sequence in a target cell, the method comprising contacting the target cell with a vector comprising:
a. i. a first pair of loxP sequences, inverted in orientation, with respect to each other;
b. a first nucleic acid encoding a first selectable marker in sense orientation, fused in frame to an miRNA sequence of interest, wherein said nucleic acid is positioned between said first pair of loxP sequences;
c. a second nucleic acid encoding a second selectable marker, in antisense orientation, said second nucleic acid is positioned between said first pair of loxP sequences, and said second nucleic acid is 3′ with regard to said first nucleic acid;
d. a second pair of loxP sequences, inverted in orientation, with respect to each other, wherein said first loxP sequenced of said second pair is positioned between said first and said second nucleic acid, and said second loxP sequence in said second pair is positioned 3′ with respect to said first pair of loxP sequences, and said second pair of loxP sequences differs from that of said first pair of loxP sequences.
wherein said cell expresses said miRNA agent, thereby reducing expression of said coding sequence and whereby, following Cre-mediated recombination in said target cell, said miRNA agent is no longer expressed, thereby being a method of conditionally expressing a coding sequence in a target cell.

It is to be understood that any embodiment, or permutation thereof, described for a method/vector/composition hereinabove, in reference to conditionally reducing expression of a coding sequence, may be applied to that of the vectors/compositions or methods of conditionally expressing a coding sequence, and represent embodiments of this invention.

According to this aspect of the invention and in another embodiment, this invention provides a method of producing an animal genetically reactivated for a coding sequence, the method comprising contacting an embryonic stem cell with a vector for conditionally expressing a coding sequence, injecting the embryonic stem cell to a blastocyst of the animal, and obtaining an animal expressing the vector, whereby, following Cre-mediated recombination in the animal, the miRNA agent is no longer expressed and the coding sequence is expressed, thereby being a method of producing an animal genetically reactivated for a coding sequence.

In another embodiment, this invention provides a method of producing an animal genetically reactivated for a coding sequence, the method comprising contacting a single cell embryo of the animal a vector for conditionally expressing a coding sequence, and obtaining an animal expressing the vector, whereby, following Cre-mediated recombination in the animal, the miRNA agent is no longer expressed and the coding sequence is expressed, thereby being a method of producing an animal genetically reactivated for a coding sequence.

In one embodiment, conditional expression of the coding sequence is accomplished at a specific developmental stage. Such expression may be accomplished, in one embodiment, via delivery of a cre recombinase to a desired cell at a specific developmental stage, or in another embodiment, the cre recombinase is present in the cell, under the control of an inducible promoter, and cre expression is induced at a specific developmental stage. In another embodiment, conditional expression of the coding sequence is accomplished in specific tissues or cells, via similar methodology, or in another embodiment, via targeted delivery of a cre recombinase to a particular cell, such as, for example via delivery in a pseudotyped viral vector, which specifically infects a desired cell type.

In another embodiment, the coding sequence for which conditional expression is desired may comprise insulins, amylases, proteases, lipases, kinases, phosphatases, glycosyl transferases, trypsinogen, chymotrypsinogen, carboxypeptidases, hormones, ribonucleases, deoxyribonucleases, triacylglycerol lipase, phospholipase A2, elastases, amylases, blood clotting factors, UDP glucuronyl transferases, ornithine transcarbamoylases, cytochrome p450 enzymes, adenosine deaminases, serum thymic factors, thymic humoral factors, thymopoietins, growth hormones, somatomedins, costimulatory factors, antibodies, colony stimulating factors, erythropoietin, epidermal growth factors, hepatic erythropoietic factors (hepatopoietin), liver-cell growth factors, interleukins, interferons, negative growth factors, fibroblast growth factors, transforming growth factors of the α family, transforming growth factors of the β family, gastrins, secretins, cholecystokinins, somatostatins, serotonins, substance P and transcription factors and enzymes (e.g., ACC synthases and oxidases, ACP desaturases and hydroxylases, ADP-glucose pyrophorylases, ATPases, alcohol dehydrogenases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, Upases, lipoxygenases, lyso/ymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases); chemokines (e.g. CXCR4, CCR5), the RNA component of telomerase, vascular endothelial growth factor (VEGF), VEGF receptor, tumor necrosis factors nuclear factor kappa B, transcription factors, cell adhesion molecules, Insulin-like growth factor, transforming growth factor beta family members, cell surface receptors, RNA binding proteins (e.g. small nucleolar RNAs, RNA transport factors), translation factors, telomerase reverse transcriptase), or combinations thereof.

In another embodiment, the coding sequence for which conditional expression is desired may comprise a tumor suppressor gene, such as, for example, APC, BRCA 1, BRCA2, MADH4, MCC, NF 1, NF2, RB 1, TP53, WTI, or combinations thereof. Conditional expression of these genes, in turn, may in one embodiment, suppress, or in another embodiment, diminish severity, or in another embodiment, prevent metastasis of a cancer.

In another embodiment, the coding sequence for which conditional expression is desired may comprise an immunomodulating protein, such as, for example, cytokines, chemokines, complement components, immune system accessory and adhesion molecules or their receptors, such as, for example, GM-CSF, IL-2, IL-12, OX40, OX40L (gp34), lymphotactin, CD40, and CD40L, interleukins 1 to 15, interferons alpha, beta or gamma, tumour necrosis factor, granulocyte-macrophage colony stimulating factor (GM-CSF), macrophage colony stimulating factor (M-CSF), granulocyte colony stimulating factor (G-CSF), chemokines such as neutrophil activating protein (NAP), macrophage chemoattractant and activating factor (MCAF), RANTES, macrophage inflammatory peptides MIP-1a and MIP-1b, complement components and their receptors, or an accessory molecule such as B7.1, B7.2, TRAP, ICAM-1, 2 or 3, cytokine receptors, OX40, OX40-ligand (gp34), or combinations thereof.

In another embodiment, the coding sequence for which conditional expression is desired may comprise a protein, which suppresses angiogenesis. Such a scenario is desirable in a number of disease states, including cancer, hemangiomas, glaucoma, and other diseases, as will be well known to one skilled in the art. In one embodiment, suppression of angiogenesis is accomplished via conditionally expressing an endostatin.

In another embodiment, the methods/vectors/compositions of this invention do not exhibit the limitation of causing constitutive gene silencing or gene expression, in all tissues. According to this aspect of the invention, the methods of this allow for regulated expression of miRNA and thereby regulated expression of a desired coding sequence.

In another embodiment, this invention provides for kits for conditional reduction of expression, or conditional expression of a coding sequence, comprising one or more containers filled with one or more of the ingredients of the aforementioned vectors, or compositions of the invention.

The vectors of the invention may be employed, in another embodiment, in combination with a non-sterile or sterile carrier or carriers for administration to cells, tissues or organisms, such as a pharmaceutical carrier suitable for administration to an individual. Such compositions comprise, for instance, a media additive or a therapeutically effective amount of a recombinant virus of the invention and a pharmaceutically acceptable carrier or excipient. Such carriers may include, but are not limited to, saline, buffered saline, dextrose, water, glycerol, and combinations thereof. The formulation should suit the mode of administration.

The vectors or compositions of the invention may be employed alone or in conjunction with other compounds, such as additional therapeutic compounds.

The pharmaceutical compositions may be administered in any effective, convenient manner including, for instance, administration by intravascular (i.v.), intramuscular (i.m.), intranasal (i.n.), subcutaneous (s.c.), oral, rectal, intravaginal delivery, or by any means in which the recombinant virus/composition can be delivered to tissue (e.g., needle or catheter). Alternatively, topical administration may be desired for insertion into epithelial cells. Another method of administration is via aspiration or aerosol formulation.

For administration to mammals, and particularly humans, it is expected that the physician will determine the actual dosage and duration of treatment, which will be most suitable for an individual and can vary with the age, weight and response of the particular individual.

The following examples are presented in order to more fully illustrate some embodiments of the invention. They should, in no way be construed, however, as limiting the scope of the invention.

### EXAMPLES

### Materials and Methods

### Generation of Constructs

To generate pFLIP, the pMIG vector (Grez et al., Proc. Natl. Acad. Sci. USA 87:9202-9206, 1990), was modified, as follows: the vector was digested with BglII and SalI, the FLIP insert replaced ires-GFP, followed by insert fill in and ligation.

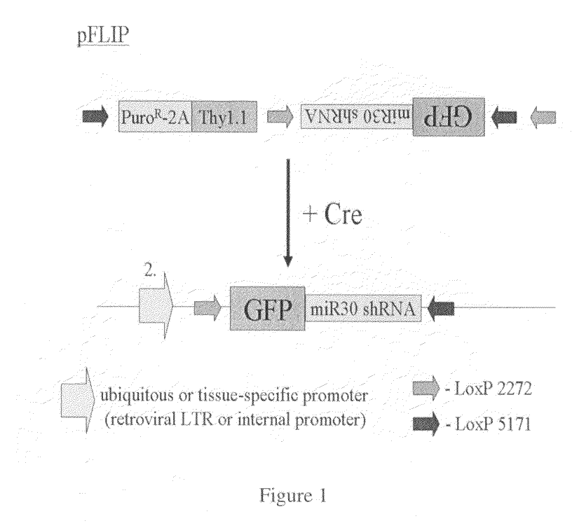

The FLIP insert comprises loxP 5171 and loxP 2272 genes, a modified puromycin resistance cassette incorporating the foot-and-mouth-disease virus (FMDV) 2A encoding sequence at its C-terminus, fused in frame to a gene encoding the Thy1.1 surface marker (See Schnutgen F, et. al., Nat Biotechnol. 2003 May; 21(5):562-5 for methods), and nucleic acids encoding miR30 and GFP, as outlined in FIG. 1.

The complete sequence of the pFLIP insert is as follows:

[SEQ ID NO: 1]

gtcgacggat ccataacttc gtataggata ccttatacga
agttatctca ggtaccgccaccatgaccga gtacaagccc
acggtgcgcc tcgccacccg cgacgacgtc
cccagggccgtacgcaccct cgccgccgcg ttcgccgact
accccgccac gcgccacacc gtcgatccggaccgccacat
cgagcgggtc accgagctgc aagaactctt cctcacgcgc
gtcgggctcgacatcggcaa ggtgtgggtc gcggacgacg
gcgccgcggt ggcggtctgg accacgccggagagcgtcga
agcgggggcg gtgttcgccg agatcggccc gcgcatggcc
gagttgagcggttcccggct ggccgcgcag caacagatgg
aaggcctcct ggcgccgcac cggcccaaggagcccgcgtg
gttcctggcc accgtcggcg tctcgcccga ccaccagggc
aagggtctgggcagcgccgt cgtgctcccc ggagtggagg
cggccgagcg cgccggggtg cccgccttcctggagacctc
cgcgccccgc aacctcccct tctacgagcg gctcggcttc
accgtcaccgccgacgtcga ggtgcccgaa ggaccgcgca
cctggtgcat gacccgcaag cccggtgccctgtacaagaa
acagaaaatt gtggcaccag tgaaacagac tttgaatttt
gaccttctcaagttggcggg agacgtcgag tccaaccctg
ggcccatgaa cccagccatc agcgtcgctctcctgctctc
agtcttgcag gtgtcccgag ggcagaaggt gaccagcctg
acagcctgcctggtgaacca aaaccttcgc ctggactgcc
gccatgagaa taacaccaag gataactccatccagcatga
gttcagcctg acccgagaga agaggaagca cgtgctctca
ggcaccctcgggatacccga gcacacgtac cgctcccgcg
tcaccctctc caaccagccc tatatcaaggtccttaccct
agccaacttc accaccaagg atgagggcga ctacttttgt
gagcttcgagtctcgggcgc gaatcccatg agctccaata
aaagtatcag tgtgtataga gacaaactggtcaagtgtgg
cggcataagc ctgctggttc agaacacatc ctggatgctg
ctgctgctgctttccctctc cctcctccaa gccctggact
tcatttctct gtgatctaga agccataacttcgtatagta
cacattatac gaagttatgt ttaaacgcat tagtcttcca
attgaaaaaagtgatttaat ttataccatt ttaattcagc
tttgtaaaaa tgtatcaaag agatagcaaggtattcagtt
ttagtaaaca agataattgc tcctaaagta gccccttgaa
ttctggttgctcgagccttc tgttgggtta acctgaagaa
gtaatcccag caagtgtttc caagatgtgcaggcaacgat
tctgtaaagt actgaagcct cattcaaaca tagtatatgt
gctgccgaagcgagcactta acaaggcttg cggccgctac
ttgtacagct cgtccatgcc gagagtgatcccggcggcgg
tcacgaactc cagcaggacc atgtgatcgc gcttctcgtt
ggggtctttgctcagggcgg actgggtgct caggtagtgg
ttgtcgggca gcagcacggg gccgtcgccgatgggggtgt
tctgctggta gtggtcggcg agctgcacgc tgccgtcctc
gatgttgtggcggatcttga agttcacctt gatgccgttc
ttctgcttgt cggccatgat atagacgttgtggctgttgt
agttgtactc cagcttgtgc cccaggatgt tgccgtcctc
cttgaagtcgatgcccttca gctcgatgcg gttcaccagg
gtgtcgccct cgaacttcac ctcggcgcgggtcttgtagt
tgccgtcgtc cttgaagaag atggtgcgct cctggacgta
gccttcgggcatggcggact tgaagaagtc gtgctgcttc
atgtggtcgg ggtagcggct gaagcactgcacgccgtagg
tcagggtggt cacgagggtg ggccagggca cgggcagctt
gccggtggtgcagatgaact tcagggtcag cttgccgtag
gtggcatcgc cctcgccctc gccggacacgctgaacttgt
ggccgtttac gtcgccgtcc agctcgacca ggatgggcac
caccccggtgaacagctcct cgcccttgct caccatggtg
gcgaccggta taacttcgta taaggtatcctatacgaagt
tatccattca ggctgtgcta gcatcaatgg catggcacaa
agcttagccataacttcgta taatgtgtac tatacgaagt
tatcccgggt taac.

The RNAi encoding sequence against firefly luciferase is as follows:

[SEQ ID NO: 2]

AAGGTATATTGCTGTTGACAGTGAGCGAGCTCCCGTGAATTGGAATCCTA
GTGAAGCCACAGATGTAGGATTCCAATTCAGCGGGAGCCTGCCTACTGCC
TCG,

and pFLIP comprising the RNAi sequence encoding sequence against firefly luciferase is as follows:

[SEQ ID NO: 3]

gtcgacggatccataacttcgtataggataccttatacgaagttatctca
ggtaccgccaccatgaccgagtacaagcccacggtgcgcctcgccacccg
cgacgacgtccccagggccgtacgcaccctcgccgccgcgttcgccgact
accccgccacgcgccacaccgtcgatccggaccgccacatcgagcgggtc
accgagctgcaagaactcttcctcacgcgcgtcgggctcgacatcggcaa
ggtgtgggtcgcggacgacggcgccgcggtggcggtctggaccacgccgg
agagcgtcgaagcgggggcggtgttcgccgagatcggcccgcgcatggcc
gagttgagcggttcccggctggccgcgcagcaacagatggaaggcctcct
ggcgccgcaccggcccaaggagcccgcgtggttcctggccaccgtcggcg
tctcgcccgaccaccagggcaagggtctgggcagcgccgtcgtgctcccc
ggagtggaggcggccgagcgcgccggggtgcccgccttcctggagacctc
cgcgccccgcaacctccccttctacgagcggctcggcttcaccgtcaccg
ccgacgtcgaggtgcccgaaggaccgcgcacctggtgcatgacccgcaag
cccggtgccctgtacaagaaacagaaaattgtggcaccagtgaaacagac
tttgaattttgaccttctcaagttggcgggagacgtcgagtccaaccctg
ggcccatgaacccagccatcagcgtcgctctcctgctctcagtcttgcag
gtgtccgagggcagaaggtgaccagcctgacagcctgcctggtgaaccaa
aaccttcgcctggactgccgccatgagaataacaccaaggataactccat
ccagcatgagttcagcctgacccgagagaagaggaagcacgtgctctcag
gcaccctcgggatacccgagcacacgtaccgctcccgcgtcaccctctcc
aaccagccctatatcaaggtccttaccctagccaacttcaccaccaagga
tgagggcgactacttttgtgagcttcgagtctcgggcgcgaatcccatga
gctccaataaaagtatcagtgtgtatagagacaaactggtcaagtgtggc
ggcataagcctgctggttcagaacacatcctggatgctgctgctgctgct
ttccctctccctcctccaagccctggacttcatttctctgtgatctagaa
gccataacttcgtatagtacacattatacgaagttatgtttaaacgcatt
agtcttccaattgaaaaaagtgatttaatttataccattttaattcagct
ttgtaaaaatgtatcaaagagatagcaaggtattcagttttagtaaacaa
gataattgctcctaaagtagccccttgaattcCGAGGCAGTAGGCAGGCT
CCCGCTGAATTGGAATCCTACATCTGTGGCTTCACTAGGATTCCAATTCA
CGGGAGCTCGCTCACTGTCAACAGCAATATACCTTctcgagccttctgtt
gggttaacctgaagaagtaatcccagcaagtgtttccaagatgtgcaggc
aacgattctgtaaagtactgaagcctcattcaaacatagtatatgtgctg
ccgaagcgagcacttaacaaggcttgcggccgctacttgtacagctcgtc
catgccgagagtgatcccggcggcggtcacgaactccagcaggaccatgt
gatcgcgcttctcgttggggtctttgctcagggcggactgggtgctcagg
tagtggttgtcgggcagcagcacggggccgtcgccgatgggggtgttctg
ctggtagtggtcggcgagctgcacgctgccgtcctcgatgttgtggcgga
tcttgaagttcaccttgatgccgttcttctgcttgtcggccatgatatat
agacgttgtggctgttgtagttgtactccagcttgtgccccaggatgttg
ccgtcctccttgaagtcgatgcccttcagctcgatgcggttcaccagggt
gtcgccctcgaacttcacctcggcgcgggtcttgtagttgccgtcgtcct
tgaagaagatggtgcgctcctggacgtagccttcgggcatggcggacttg
aagaagtcgtgctgcttcatgtggtcggggtagcggctgaagcactgcac
gccgtaggtcagggtggtcacgagggtgggccagggcacgggcagcttgc
cggtggtgcagatgaacttcagggtcagcttgccgtaggtggcatcgccc
tcgccctcgccggacacgctgaacttgtggccgtttacgtcgccgtccag
ctcgaccaggatgggcaccaccccggtgaacagctcctcgcccttgctca
ccatggtggcgaccggtataacttcgtataaggtatcctatacgaagtta
tccattcaggctgtgctagcatcaatggcatggcacaaagcttagccata
acttcgtataatgtgtactatacgaagttatcccgggttaac.

MSCV comprising the pFLIP insert with RNAi to Luciferase is as follows (FLIP insert in lowercase):

[SEQ ID NO: 4]

TGAAAGACCCCACCTGTAGGTTTGGCAAGCTAGCTTAAGTAACGCCATTT
TGCAAGGCATGGAAAATACATAACTGAGAATAGAGAAGTTCAGATCAAGG
TTAGGAACAGAGAGACAGCAGAATATGGGCCAAACAGGATATCTGTGGTA
AGCAGTTCCTGCCCCGGCTCAGGGCCAAGAACAGATGGTCCCCAGATGCG
GTCCCGCCCTCAGCAGTTTCTAGAGAACCATCAGATGTTTCCAGGGTGCC
CCAAGGACCTGAAAATGACCCTGTGCCTTATTTGAACTAACCAATCAGTT
CGCTTCTCGCTTCTGTTCGCGCGCTTCTGCTCCCCGAGCTCAATAAAAGA
GCCCACAACCCCTCACTCGGCGCGCCAGTCCTCCGATAGACTGCGTCGCC
CGGGTACCCGTATTCCCAATAAAGCCTCTTGCTGTTTGCATCCGAATCGT
GGACTCGCTGATCCTTGGGAGGGTCTCCTCAGATTGATTGACTGCCCACC
TCGGGGGTCTTTCATTTGGAGGTTCCACCGAGATTTGGAGACCCCTGCCT
AGGGACCACCGACCCCCCCGCCGGGAGGTAAGCTGGCCAGCGGTCGTTTC
GTGTCTGTCTCTGTCTTTGTGCGTGTTTGTGCCGGCATCTAATGTTTGCG
CCTGCGTCTGTACTAGTTAGCTAACTAGCTCTGTATCTGGCGGACCCGTG
GTGGAACTGACGAGTTCTGAACACCCGGCCGCAACCCTGGGAGACGTCCC
AGGGACTTTGGGGGCCGTTTTTGTGGCCCGACCTGAGGAAGGGAGTCGAT
GTGGAATCCGACCCCGTCAGGATATGTGGTTCTGGTAGGAGACGAGAACC
TAAAACAGTTCCCGCCTCCGTCTGAATTTTTGCTTTCGGTTTGGAACCGA
AGCCGCGCGTCTTGTCTGCTGCAGCGCTGCAGCATCGTTCTGTGTTGTCT
CTGTCTGACTGTGTTTCTGTATTTGTCTGAAAATTAGGGCCAGACTGTTA
CCACTCCCTTAAGTTTGACCTTAGGTCACTGGAAAGATGTCGAGCGGATC
GCTCACAACCAGTCGGTAGATGTCAAGAAGAGACGTTGGGTTACCTTCTG
CTCTGCAGAATGGCCAACCTTTAACGTCGGATGGCCGCGAGACGGCACCT
TTAACCGAGACCTCATCACCCAGGTTAAGATCAAGGTCTTTTCACCTGGC
CCGCATGGACACCCAGACCAGGTCCCCTACATCGTGACCTGGGAAGCCTT
GGCTTTTGACCCCCCTCCCTGGGTCAAGCCCTTTGTACACCCTAAGCCTC
CGCCTCCTCTTCCTCCATCCGCCCCGTCTCTCCCCCTTGAACCTCCTCGT
TCGACCCCGCCTCGATCCTCCCTTTATCCAGCCCTCACTCCTTCTCTAGG
CGCCGGAATTAGAtccataacttcgtataggataccttatacgaagttat
ctcaggtaccgccaccatgaccgagtacaagcccacggtgcgcctcgcca
cccgcgacgacgtccccagggccgtacgcaccctcgccgccgcgttcgcc
gactaccccgccacgcgccacaccgtcgatccggaccgccacatcgagcg
ggtcaccgagctgcaagaactcttcctcacgcgcgtcgggctcgacatcg
gcaaggtgtgggtcgcggacgacggcgccgcggtggcggtctggaccacg
ccggagagcgtcgaagcgggggcggtgttcgccgagatcggcccgcgcat
ggccgagttgagcggttcccggctggccgcgcagcaacagatggaaggcc
tcctggcgccgcaccggcccaaggagcccgcgtggttcctggccaccgtc
ggcgtctcgcccgaccaccagggcaagggtctgggcagcgccgtcgtgct
ccccggagtggaggcggccgagcgcgccggggtgcccgccttcctggaga
cctccgcgccccgcaacctccccttctacgagcggctcggcttcaccgtc
accgccgacgtcgaggtgcccgaaggaccgcgcacctggtgcatgacccg
caagcccggtgccctgtacaagaaacagaaaattgtggcaccagtgaaac
agactttgaattttgaccttctcaagttggcgggagacgtcgagtccaac
cctgggcccatgaacccagccatcagcgtcgctctcctgctctcagtctt
gcaggtgtcccgagggcagaaggtgaccagcctgacagcctgcctggtga
accaaaaccttcgcctggactgccgccatgagaataacaccaaggataac
tccatccagcatgagttcagcctgacccgagagaagaggaagcacgtgct
ctcaggcaccctcgggatacccgagcacacgtaccgctcccgcgtcaccc
tctccaaccagccctatatcaaggtccttaccctagccaacttcaccacc
aaggatgagggcgactacttttgtgagcttcgagtctcgggcgcgaatcc
catgagctccaataaaagtatcagtgtgtatagagacaaactggtcaagt
gtggcggcataagcctgctggttcagaacacatcctggatgctgctgctg
ctgctttccctctccctcctccaagccctggacttcatttctctgtgatc
tagaagccataacttcgtatagtacacattatacgaagttatgtttaaac
gcattagtcttccaattgaaaaaagtgatttaatttataccattttaatt
cagctttgtaaaaatgtatcaaagagtagcaaggtattcagttttagtaa
caagataattgctcctaaagtagccccttgaattcCGAGGCAGTAGGCAG
GCTCCCGCTGAATTGGAATCCTACATCTGTGGCTTCACTAGGATTCCAAT
TCACGGGAGCTCGCTCACTGTCAACAGCAATATACCTTctcgagccttct
gttgggttaacctgaagaagtaatcccagcaagtgtttccaagatgtgca
ggcaacgattctgtaaagtactgaagcctcattcaaacatagtatatgtg
ctgccgaagcgagcacttaacaaggcttgcggccgctacttgtacagctc
gtccatgccgagagtgatcccggcggcggtcacgaactccagcaggacca
tgtgatcgcgcttctcgttggggtctttgctcagggcggactgggtgctc
aggtagtggttgtcgggcagcagcacggggccgtcgccgatgggggtgtt
ctgctggtagtggtcggcgagctgcacgctgccgtcctcgatgttgtggc
ggatcttgaagttcaccttgatgccgttcttctgcttgtcggccatgata
tagacgttgtggctgttgtagttgtactccagcttgtgccccaggatgtt
gccgtcctccttgaagtcgatgcccttcagctcgatgcggttcaccaggg
tgtcgccctcgaacttcacctcggcgcgggtcttgtagttgccgtcgtcc
ttgaagaagatggtgcgctcctggacgtagccttcgggcatggcggactt
gaagaagtcgtgctgcttcatgtggtcggggtagcggctgaagcactgca
cgccgtaggtcagggtggtcacgagggtgggccagggcacgggcagcttg
ccggtggtgcagatgaacttcagggtcagcttgccgtaggtggcatcgcc
ctcgccctcgccggacacgctgaacttgtggccgtttacgtcgccgtcca
gctcgaccaggatgggcaccaccccggtgaacagctcctcgcccttgctc
accatggtggcgaccggtataacttcgtataaggtatcctatacgaagtt
atccattcaggctgtgctagcatcaatggcatggcacaaagcttagccat
aacttcgtataatgtgtactatacgaagttatcccgggttAAACGACCTG
CAGCCAAGCTTATCGATAAAATAAAAGATTTTATTTAGTCTCCAGAAAAA
GGGGGGAATGAAAGACCCCACCTGTAGGTTTGGCAAGCTAGCTTAAGTAA
CGCCATTTTGCAAGGCATGGAAAATACATAACTGAGAATAGAGAAGTTCA
GATCAAGGTTAGGAACAGAGAGACAGCAGAATATGGGCCAAACAGGATAT
CTGTGGTAAGCAGTTCCTGCCCCGGCTCAGGGCCAAGAACAGATGGTCCC
CAGATGCGGTCCCGCCCTCAGCAGTTTCTAGAGAACCATCAGATGTTTCC
AGGGTGCCCCAAGGACCTGAAAATGACCCTGTGCCTTATTTGAACTAACC
AATCAGTTCGCTTCTCGCTTCTGTTCGCGCGCTTCTGCTCCCCGAGCTCA
ATAAAAGAGCCCACAACCCCTCACTCGGCGCGCCAGTCCTCCGATAGACT
GCGTCGCCCGGGTACCCGTGTATCCAATAAACCCTCTTGCAGTTGCATCC
GACTTGTGGTCTCGCTGTTCCTTGGGAGGGTCTCCTCTGAGTGATTGACT
ACCCGTCAGCGGGGGTCTTTCATGGGTAACAGTTTCTTGAAGTTGGAGAA
CAACATTCTGAGGGTAGGAGTCGAATATTAAGTAATCCTGACTCAATTAG
CCACTGTTTTGAATCCACATACTCCAATACTCCTGAAATAGTTCATTATG
GACAGCGCAGAAGAGCTGGGGAGAATTAATTCGTAATCATGGTCATAGCT
GTTTCCTGTGTGAAATTGTTATCCGCTCACAATTCCACACAACATACGAG
CCGGAAGCATAAAGTGTAAAGCCTGGGGTGCCTAATGAGTGAGCTAACTC
ACATTAATTGCGTTGCGCTCACTGCCCGCTTTCCAGTCGGGAAACCTGTC
GTGCCAGCTGCATTAATGAATCGGCCAACGCGCGGGGAGAGGCGGTTTGC
GTATTGGGCGCTCTTCCGCTTCCTCGCTCACTGACTCGCTGCGCTCGGTC
GTTCGGCTGCGGCGAGCGGTATCAGCTCACTCAAAGGCGGTAATACGGTT
ATCCACAGAATCAGGGGATAACGCAGGAAAGAACATGTGAGCAAAAGGCC
AGCAAAAGGCCAGGAACCGTAAAAAGGCCGCGTTGCTGGCGTTTTTCCAT
AGGCTCCGCCCCCCTGACGAGCATCACAAAAATCGACGCTCAAGTCAGAG
GTGGCGAAACCCGACAGGACTATAAAGATACCAGGCGTTTCCCCCTGGAA
GCTCCCTCGTGCGCTCTCCTGTTCCGACCCTGCCGCTTACCGGATACCTG
TCCGCCTTTCTCCCTTCGGGAAGCGTGGCGCTTTCTCATAGCTCACGCTG
TAGGTATCTCAGTTCGGTGTAGGTCGTTCGCTCCAAGCTGGGCTGTGTGC
ACGAACCCCCCGTTCAGCCCGACCGCTGCGCCTTATCCGGTAACTATCGT
CTTGAGTCCAACCCGGTAAGACACGACTTATCGCCACTGGCAGCAGCCAC
TGGTAACAGGATTAGCAGAGCGAGGTATGTAGGCGGTGCTACAGAGTTCT
TGAAGTGGTGGCCTAACTACGGCTACACTAGAAGGACAGTATTTGGTATC
TGCGCTCTGCTGAAGCCAGTTACCTTCGGAAAAAGAGTTGGTAGCTCTTG
ATCCGGCAAACAAACCACCGCTGGTAGCGGTGGTTTTTTTGTTTGCAAGC
AGCAGATTACGCGCAGAAAAAAAGGATCTCAAGAAGATCCTTTGATCTTT
TCTACGGGGTCTGACGCTCAGTGGAACGAAAACTCACGTTAAGGGATTTT
GGTCATGAGATTATCAAAAAGGATCTTCACCTAGATCCTTTTAAATTAAA
AATGAAGTTTTAAATCAATCTAAAGTATATATGAGTAAACTTGGTCTGAC
AGTTACCAATGCTTAATCAGTGAGGCACCTATCTCAGCGATCTGTCTATT
TCGTTCATCCATAGTTGCCTGACTCCCCGTCGTGTAGATAACTACGATAC
GGGAGGGCTTACCATCTGGCCCCAGTGCTGCAATGATACCGCGAGACCCA
CGCTCACCGGCTCCAGATTTATCAGCAATAAACCAGCCAGCCGGAAGGGC
CGAGCGCAGAAGTGGTCCTGCAACTTTATCCGCCTCCATCCAGTCTATTA
ATTGTTGCCGGGAAGCTAGAGTAAGTAGTTCGCCAGTTAATAGTTTGCGC
AACGTTGTTGCCATTGCTACAGGCATCGTGGTGTCACGCTCGTCGTTTGG
TATGGCTTCATTCAGCTCCGGTTCCCAACGATCAAGGCGAGTTACATGAT
CCCCCATGTTGTGCAAAAAAGCGGTTAGCTCCTTCGGTCCTCCGATCGTT
GTCAGAAGTAAGTTGGCCGCAGTGTTATCACTCATGGTTATGGCAGCACT
GCATAATTCTCTTACTGTCATGCCATCCGTAAGATGCTTTTCTGTGACTG
GTGAGTACTCAACCAAGTCATTCTGAGAATAGTGTATGCGGCGACCGAGT
TGCTCTTGCCCGGCGTCAATACGGGATAATACCGCGCCACATAGCAGAAC
TTTAAAAGTGCTCATCATTGGAAAACGTTCTTCGGGGCGAAAACTCTCAA
GGATCTTACCGCTGTTGAGATCCAGTTCGATGTAACCCACTCGTGCACCC
AACTGATCTTCAGCATCTTTTACTTTCACCAGCGTTTCTGGGTGAGCAAA
AACAGGAAGGCAAAATGCCGCAAAAAAGGGAATAAGGGCGACACGGAAAT
GTTGAATACTCATACTCTTCCTTTTTCAATATTATTGAAGCATTTATCAG
GGTTATTGTCTCATGAGCGGATACATATTTGAATGTATTTAGAAAAATAA
ACAAATAGGGGTTCCGCGCACATTTCCCCGAAAAGTGCCACCTGACGTCT
AAGAAACCATTATTATCATGACATTAACCTATAAAAATAGGCGTATCACG
AGGCCCTTTCGTCTCGCGCGTTTCGGTGATGACGGTGAAAACCTCTGACA
CATGCAGCTCCCGGAGACGGTCACAGCTTGTCTGTAAGCGGATGCCGGGA
GCAGACAAGCCCGTCAGGGCGCGTCAGCGGGTTGGGCGGGTGTCGGGGCT
GGCTTAACTATGCGGCATCAGAGCAGATTGTACTGAGAGTGCACCATATG
CGGTGTGAAATACCGCACAGATGCGTAAGGAGAAAATACCGCATCAGGCG
CCATTCGCCATTCAGGCTGCGCAACTGTTGGGAAGGGCGATCGGTGCGGG
CCTCTTCGCTATTACGCCAGCTGGCGAAAGGGGGATGTGCTGCAAGGCGA
TTAAGTTGGGTAACGCCAGGGTTTTCCCAGTCACGACGTTGTAAAACGAC
GGCGCAAGGAAGCAGCCCAGTAGTAGGTTGAGGCCGTTGAGCACCGCCGC
CGCAAGGAATGGTGCATGCAAGGAGATGGCGCCCAACAGTCCCCCGGCCA
CGGGGCCTGCCACCATACCCACGCCGAAACAAGCGCTCATGAGCCCGAAG
TGGCGAGCCCGATCTTCCCCATCGGTGATGTCGGCGATATAGGCGCCAGC
AACCGCACCTGTGGCGCCGGTGATGCCGGCCACGATGCGTCCGGCGTAGA
GGCGATTAGTCCAATTTGTTAAAGACAGGATATCAGTGGTCCAGGCTCTA
GTTTTGACTCAACAATATCACCAGCTGAAGCCTATAGAGTACGAGCCATA
GATAAAATAAAAGATTTTATTTAGTCTCCAGAAAAAGGGGGGAA.

The RNAi encoding sequence against p53 is as follows:

[SEQ ID NO: 5]

AAGGTATATTGCTGTTGACAGTGAGCGCCCACTACAAGTACATGTGTAAT
GTGAAGCCACAGATGTATTACACATGTACTTGTAGTGGATGCCTACTGCC
TCG.

The pFLIP insert comprising an RNAi to p53 has a nucleic acid sequence as follows (microRNA-short hairpin to p53 in upper case):

[SEQ ID NO: 6]

tccataacttcgtataggataccttatacgaagttatctcaggtaccgcc
accatgaccgagtacaagcccacggtgcgcctcgccacccgcgacgacgt
ccccagggccgtacgcaccctcgccgccgcgttcgccgactaccccgcca
cgcgccacaccgtcgatccggaccgccacatcgagcgggtcaccgagctg
caagaactcttcctcacgcgcgtcgggctcgacatcggcaaggtgtgggt
cgcggacgacggcgccgcggtggcggtctggaccacgccggagagcgtcg
aagcgggggcggtgttcgccgagatcggcccgcgcatggccgagttgagc
ggttcccggctggccgcgcagcaacagatggaaggcctcctggcgccgca
ccggcccaaggagcccgcgtggttcctggccaccgtcggcgtctcgcccg
accaccagggcaagggtctgggcagcgccgtcgtgctccccggagtggag
gcggccgagcgcgccggggtgcccgccttcctggagacctccgcgccccg
caacctccccttctacgagcggctcggcttcaccgtcaccgccgacgtcg
aggtgcccgaaggaccgcgcacctggtgcatgacccgcaagcccggtgcc
ctgtacaagaaacagaaaattgtggcaccagtgaaacagactttgaattt
tgaccttctcaagttggcgggagacgtcgagtccaaccctgggcccatga
acccagccatcagcgtcgctctcctgctctcagtcttgcaggtgtcccga
gggcagaaggtgaccagcctgacagcctgcctggtgaaccaaaaccttcg
cctggactgccgccatgagaataacaccaaggataactccatccagcatg
agttcagcctgacccgagagaagaggaagcacgtgctctcaggcaccctc
gggatacccgagcacacgtaccgctcccgcgtcaccctctccaaccagcc
ctatatcaaggtccttaccctagccaacttcaccaccaaggatgagggcg
actacttttgtgagcttcgagtctcgggcgcgaatcccatgagctccaat
aaaagtatcagtgtgtatagagacaaactggtcaagtgtggcggcataag
cctgctggttcagaacacatcctggatgctgctgctgctgctttccctct
ccctcctccaagccctggacttcatttctctgtgatctagaagccataac
ttcgtatagtacacattatacgaagttatGTTTAAACGCATTAGTCTTCC
AATTGAAAAAAGTGATTTAATTTATACCATTTAATTCAGCTTTGTAAAAA
TGTATCAAAGAGATAGCAAGGTATTCAGTTTTAGTAAACAAGATAATTGC
TCCTAAAGTAGCCCCTTGAATTCCGAGGCAGTAGGCATCCACTACAAGTA
CATGTGTAATACATCTGTGGCTTCACTATTACACATGTACTTGTAGTGGG
CGCTCACTGTCAACAGCAATATACCTTCTCGAGCCTTCTGTTGGGTTAAC
CTGAAGAAGTAATCCCAGCAAGTGTTTCCAAGATGTGCAGGCAACGATTC
TGTAAAGTACTGAAGCCTCATTCAAACATAGTATATGTGCTGCCGAAGCG
AGCACTTAACAAGGCTTGCGGCCGCtacttgtacagctcgtccatgccga
gagtgatcccggcggcggtcacgaactccagcaggaccatgtgatcgcgc
ttctcgttggggtctttgctcagggcggactgggtgctcaggtagtggtt
gtcgggcagcagcacggggccgtcgccgatgggggtgttctgctggtagt
ggtcggcgagctgcacgctgccgtcctcgatgttgtggcggatcttgaag
ttcaccttgatgccgttcttctgcttgtcggccatgatatagacgttgtg
gctgttgtagttgtactccagcttgtgccccaggatgttgccgtcctcct
tgaagtcgatgcccttcagctcgatgcggttcaccagggtgtcgccctcg
aacttcacctcggcgcgggtcttgtagttgccgtcgtccttgaagaagat
ggtgcgctcctggacgtagccttcgggcatggcggacttgaagaagtcgt
gctgcttcatgtggtcggggtagcggctgaagcactgcacgccgtaggtc
agggtggtcacgagggtgggccagggcacgggcagcttgccggtggtgca
gatgaacttcagggtcagcttgccgtaggtggcatcgccctcgccctcgc
cggacacgctgaacttgtggccgtttacgtcgccgtccagctcgaccagg
atgggcaccaccccggtgaacagctcctcgcccttgctcaccatggtggc
gaccggtataacttcgtataaggtatcctatacgaagttatccattcagg
ctgtgctagcatcaatggcatggcacaaagcttagccataacttcgtata
atgtgtactatacgaagttatcccgggtt.

The MSCV FLIP-p53 construct has a nucleotide sequence as follows (FLIP insert begins base 1411 lower case, microRNA-short hairpin to p53 in upper case):

[SEQ ID NO:7]

TGAAAGACCCCACCTGTAGGTTTGGCAAGCTAGCTTAAGTAACGCCATTT
TGCAAGGCATGGAAAATACATAACTGAGAATAGAGAAGTTCAGATCAAGG
TTAGGAACAGAGAGACAGCAGAATATGGGCCAAACAGGATATCTGTGGTA
AGCAGTTCCTGCCCCGGCTCAGGGCCAAGAACAGATGGTCCCCAGATGCG
GTCCCGCCCTCAGCAGTTTCTAGAGAACCATCAGATGTTTCCAGGGTGCC
CCAAGGACCTGAAAATGACCCTGTGCCTTATTTGAACTAACCAATCAGTT
CGCTTCTCGCTTCTGTTCGCGCGCTTCTGCTCCCCGAGCTCAATAAAAGA
GCCCACAACCCCTCACTCGGCGCGCCAGTCCTCCGATAGACTGCGTCGCC
CGGGTACCCGTATTCCCAATAAAGCCTCTTGCTGTTTGCATCCGAATCGT
GGACTCGCTGATCCTTGGGAGGGTCTCCTCAGATTGATTGACTGCCCACC
TCGGGGGTCTTTCATTTGGAGGTTCCACCGAGATTTGGAGACCCCTGCCT
AGGGACCACCGACCCCCCCGCCGGGAGGTAAGCTGGCCAGCGGTCGTTTC
GTGTCTGTCTCTGTCTTTGTGCGTGTTTGTGCCGGCATCTAATGTTTGCG
CCTGCGTCTGTACTAGTTAGCTAACTAGCTCTGTATCTGGCGGACCCGTG
GTGGAACTGACGAGTTCTGAACACCCGGCCGCAACCCTGGGAGACGTCCC
AGGGACTTTGGGGGCCGTTTTTGTGGCCCGACCTGAGGAAGGGAGTCGAT
GTGGAATCCGACCCCGTCAGGATATGTGGTTCTGGTAGGAGACGAGAACC
TAAAACAGTTCCCGCCTCCGTCTGAATTTTTGCTTTCGGTTTGGAACCGA
AGCCGCGCGTCTTGTCTGCTGCAGCGCTGCAGCATCGTTCTGTGTTGTCT
CTGTCTGACTGTGTTTCTGTATTTGTCTGAAAATTAGGGCCAGACTGTTA
CCACTCCCTTAAGTTTGACCTTAGGTCACTGGAAAGATGTCGAGCGGATC
GCTCACAACCAGTCGGTAGATGTCAAGAAGAGACGTTGGGTTACCTTCTG
CTCTGCAGAATGGCCAACCTTTAACGTCGGATGGCCGCGAGACGGCACCT
TTAACCGAGACCTCATCACCCAGGTTAAGATCAAGGTCTTTTCACCTGGC
CCGCATGGACACCCAGACCAGGTCCCCTACATCGTGACCTGGGAACCCTT
GGCTTTTGACCCCCCTCCCTGGGTCAAGCCCTTTGTACACCCTAAGCCTC
CGCCTCCTCTTCCTCCATCCGCCCCGTCTCTCCCCCTTGAACCTCCTCGT
TCGACCCCGCCTCGATCCTCCCTTTATCCAGCCCTCACTCCTTCTCTAGG
CGCCGGAATTAGAtccataacttcgtataggataccttatacgaagttat
ctcaggtaccgccaccatgaccgagtacaagcccacggtgcgcctcgcca
cccgcgacgacgtccccagggccgtacgcaccctcgccgccgcgttcgcc
gactaccccgccacgcgccacaccgtcgatccggaccgccacatcgagcg
ggtcaccgagctgcaagaactcttcctcacgcgcgtcgggctcgacatcg
gcaaggtgtgggtcgcggacgacggcgccgcggtggcggtctggaccacg
ccggagagcgtcgaagcgggggcggtgttcgccgagatcggcccgcgcat
ggccgagttgagcggttcccggctggccgcgcagcaacagatggaaggcc
tcctggcgccgcaccggcccaaggagcccgcgtggttcctggccaccgtc
ggcgtctcgcccgaccaccagggcaagggtctgggcagcgccgtcgtgct
ccccggagtggaggcggccgagcgcgccggggtgcccgccttcctggaga
cctccgcgccccgcaacctccccttctacgagcggctcggcttcaccgtc
accgccgacgtcgaggtgcccgaaggaccgcgcacctggtgcatgacccg
caagcccggtgccctgtacaagaaacagaaaattgtggcaccagtgaaac
agactttgaattttgaccttcttaagttggcgggagacgtcgagtccaac
cctgggcccatgaacccagccatcagcgtcgctctcctgctctcagtctt
gcaggtgtcccgagggcagaaggtgaccagcctgacagcctgcctggtga
accaaaaccttcgcctggactgccgccatgagaataacaccaaggataac
tccatccagcatgagttcagcctgacccgagagaagaggaagcacgtgct
ctcaggcaccctcgggatacccgagcacacgtaccgctcccgcgtcaccc
tctccaaccagccctatatcaaggtccttaccctagccaacttcaccacc
aaggatgagggcgactacttttgtgagcttcgagtctcgggcgcgaatcc
catgagctccaataaaagtatcagtgtgtatagagacaaactggtcaagt
gtggcggcataagcctgctggttcagaacacatcctggatgctgctgctg
ctgctttccctctccctcctccaagccctggacttcatttctctgtgatc
tagaagccataacttcgtatagtacacattatacgaagttatGTTTAAAC
GCATTAGTCTTCCAATTGAAAAAAGTGATTTAATTTATACCATTTTAATT
CAGCTTTGTAAAAATGTATCAAAGAGATAGCAAGGTATTCAGTTTTAGTA
AACAAGATAATTGCTCCTAAAGTAGCCCCTTGAATTCCGAGGCAGTAGGC
ATCCACTACAAGTACATGTGTAATACATCTGTGGCTTCACTATTACACAT
GTACTTGTAGTGGGCGCTCACTGTCAACAGCAATATACCTTCTCGAGCCT
TCTGTTGGGTTAACCTGAAGAAGTAATCCCAGCAAGTGTTTCCAAGATGT
GCAGGCAACGATTCTGTAAAGTACTGAAGCCTCATTCAAACATAGTATAT
GTGCTGCCGAAGCGAGCACTTAACAAGGCTTGCGGCCGCtacttgtacag
ctcgtccatgccgagagtgatcccggcggcggtcacgaactccagcagga
ccatgtgatcgcgcttctcgttggggtctttgctcagggcggactgggtg
ctcaggtagtggttgtcgggcagcagcacggggccgtcgccgatgggggt
gttctgctggtagtggtcggcgagctgcacgctgccgtcctcgatgttgt
ggcggatcttgaagttcaccttgatgccgttcttctgcttgtcggccatg
atatagacgttgtggctgttgtagttgtactccagcttgtgccccaggat
gttgccgtcctccttgaagtcgatgcccttcagctcgatgcggttcacca
gggtgtcgccctcgaacttcacctcggcgcgggtcttgtagttgccgtcg
tccttgaagaagatggtgcgctcctggacgtagccttcgggcatggcgga
cttgaagaagtcgtgctgcttcatgtggtcggggtagcggctgaagcact
gcacgccgtaggtcagggtggtcacgagggtgggccagggcacgggcagc
ttgccggtggtgcagatgaacttcagggtcagcttgccgtaggtggcatc
gccctcgccctcgccggacacgctgaacttgtggccgtttacgtcgccgt
ccagctcgaccaggatgggcaccaccccggtgaacagctcctcgcccttg
ctcaccatggtggcgaccggtataacttcgtataaggtatcctatacgaa
gttatccattcaggctgtgctagcatcaatggcatggcacaaagcttagc
cataacttcgtataatgtgtactatacgaagttatcccgggttAAACGAC
CTGCAGCCAAGCTTATCGATAAAATAAAAGATTTTATTTAGTCTCCAGAA
AAAGGGGGGAATGAAAGACCCCACCTGTAGGTTTGGCAAGCTAGCTTAAG
TAACGCCATTTTGCAAGGCATGGAAAATACATAACTGAGAATAGAGAAGT
TCAGATCAAGGTTAGGAACAGAGAGACAGCAGAATATGGGCCAAACAGGA
TATCTGTGGTAAGCAGTTCCTGCCCCGGCTCAGGGCCAAGAACAGATGGT
CCCCAGATGCGGTCCCGCCCTCAGCAGTTTCTAGAGAACCATCAGATGTT
TCCAGGGTGCCCCAAGGACCTGAAAATGACCCTGTGCCTTATTTGAACTA
ACCAATCAGTTCGCTTCTCGCTTCTGTTCGCGCGCTTCTGCTCCCCGAGC
TCAATAAAAGAGCCCACAACCCCTCACTCGGCGCGCCAGTCCTCCGATAG
ACTGCGTCGCCCGGGTACCCGTGTATCCAATAAACCCTCTTGCAGTTGCA
TCCGACTTGTGGTCTCGCTGTTCCTTGGGAGGGTCTCCTCTGAGTGATTG
ACTACCCGTCAGCGGGGGTCTTTCATGGGTAACAGTTTCTTGAAGTTGGA
GAACAACATTCTGAGGGTAGGAGTCGAATATTAAGTAATCCTGACTCAAA
TTAGCCACTGTTTTGAATCCACATACTCCAATACTCCTGAAATAGTTCAT
TATGGACAGCGCAGAAGAGCTGGGGAGAATTAATTCGTAATCATGGTCAT
AGCTGTTTCCTGTGTGAAATTGTTATCCGCTCACAATTCCACACAACATA
CGAGCCGGAAGCATAAAGTGTAAAGCCTGGGGTGCCTAATGAGTGAGCTA
ACTCACATTAATTGCGTTGCGCTCACTGCCCGCTTTCCAGTCGGGAAACC
TGTCGTGCCAGCTGCATTAATGAATCGGCCAACGCGCGGGGAGAGGCGGT
TGCGTATTGCGCGCTCTTCCGCTTCCTCGCTCACTGACTCGCTGCGCTCG
GTCGTTCGGCTGCGGCGAGCGGTATCAGCTCACTCAAAGGCGGTAATACG
GTTATCCACAGAATCAGGGGATAACGCAGGAAAGAACATGTGAGCAAAAG
GCCAGCAAAAGGCCAGGAACCGTAAAAAGGCCGCGTTGCTGGCGTTTTTC
CATAGGCTCCGCCCCCCTGACGAGCATCACAAAAATCGACGCTCAAGTCA
GAGGTGGCGAAACCCGACAGGACTATAAAGATACCAGGCGTTTCCCCCTG
GAAGCTCCCTCGTGCGCTCTCCTGTTCCGACCCTGCCGCTTACCGGATAC
CTGTCCGCCTTTCTCCCTTCGGGAAGCGTGGCGCTTTCTCATAGCTCACG
CTGTAGGTATCTCAGTTCGGTGTAGGTCGTTCGCTCCAAGCTGGGCTGTG
TGCACGAACCCCCCGTTCAGCCCGACCGCTGCGCCTTATCCGGTAACTAT
CGTCTTGAGTCCAACCCGGTAAGACACGACTTATCGCCACTGGCAGCAGC
CACTGGTAACAGGATTAGCAGAGCGAGGTATGTAGGCGGTGCTACAGAGT
TCTTGAAGTGGTGGCCTAACTACGGCTACACTAGAAGGACAGTATTTGGT
ATCTGCGCTCTGCTGAAGCCAGTTACCTTCGGAAAAAGAGTTGGTAGCTC
TTGATCCGGCAAACAAACCACCGCTGGTAGCGGTGGTTTTTTTGTTTGCA
AGCAGCAGATTACGCGCAGAAAAAAAGGATCTCAAGAAGATCCTTTGATC
TTTTCTACGGGGTCTGACGCTCAGTGGAACGAAAACTCACGTTAAGGGAT
TTTGGTCATGAGATTATCAAAAAGGATCTTCACCTAGATCCTTTTAAATT
AAAAATGAAGTTTTAAATCAATCTAAAGTATATATGAGTAAACTTGGTCT
GACAGTTACCAATGCTTAATCAGTGAGGCACCTATCTCAGCGATCTGTCT
ATTTCGTTCATCCATAGTTGCCTGACTCCCCGTCGTGTAGATAACTACGA
TACGGGAGGGCTTACCATCTGGCCCCAGTGCTGCAATGATACCGCGAGAC
CCACGCTCACCGGCTCCAGATTTATCAGCAATAAACCAGCCAGCCGGAAG
GGCCGAGCGCAGAAGTGGTCCTGCAACTTTATCCGCCTCCATCCAGTCTA
TTAATTGTTGCCGGGAAGCTAGAGTAAGTAGTTCGCCAGTTAATAGTTTG
CGCAACGTTGTTGCCATTGCTACAGGCATCGTGGTGTCACGCTCGTCGTT
TGGTATGGCTTCATTCAGCTCCGGTTCCCAACGATCAAGGCGAGTTACAT
GATCCCCCATGTTGTGCAAAAAAGCGGTTAGCTCCTTCGGTCCTCCGATC
GTTGTCAGAAGTAAGTTGGCCGCAGTGTTATCACTCATGGTTATGGCAGC
ACTGCATAATTCTCTTACTGTCATGCCATCCGTAAGATGCTTTTCTGTGA
CTGGTGAGTACTCAACCAAGTCATTCTGAGAATAGTGTATGCGGCGACCG
AGTTGCTCTTGCCCGGCGTCAATACGGGATAATACCGCGCCACATAGCAG
AACTTTAAAAGTGCTCATCATTGGAAAACGTTCTTCGGGGCGAAAACTCT
CAAGGATCTTACCGCTGTTGAGATCCAGTTCGATGTAACCCACTCGTGCA
CCCAACTGATCTTCAGCATCTTTTACTTTCACCAGCGTTTCTGGGTGAGC
AAAAACAGGAAGGCAAAATGCCGCAAAAAAGGGAATAAGGGCGACACGGA
AATGTTGAATACTCATACTCTTCCTTTTTCAATATTATTGAAGCATTTAT
CAGGGTTATTGTCTCATGAGCGGATACATATTTGAATGTATTTAGAAAAA
TAAACAAATAGGGGTTCCGCGCACATTTCCCCGAAAAGTGCCACCTGACG
TCTAAGAAACCATTATTATCATGACATTAACCTATAAAAATAGGCGTATC
ACGAGGCCCTTTCGTCTCGCGCGTTTCGGTGATGACGGTGAAAACCTCTG
ACACATGCAGCTCCCGGAGACGGTCACAGCTTGTCTGTAAGCGGATGCCG
GGAGCAGACAAGCCCGTCAGGGCGCGTCAGCGGGTGTTGGCGGGTGTCGG
GGCTGGCTTAACTATGCGGCATCAGAGCAGATTGTACTGAGAGTGCACCA
TATGCGGTGTGAAATACCGCACAGATGCGTAAGGAGAAAATACCGCATCA
GGCGCCATTCGCCATTCAGGCTGCGCAACTGTTGGGAAGGGCGATCGGTG
CGGGCCTCTTCGCTATTACGCCAGCTGGCGAAAGGGGGATGTGCTGCAAG
GCGATTAAGTTGGGTAACGCCAGGGTTTTCCCAGTCACGACGTTGTAAAA
CGACGGCGCAAGGAAGCAGCCCAGTAGTAGGTTGAGGCCGTTGAGCACCG
CCGCCGCAAGGAATGGTGCATGCAAGGAGATGGCGCCCAACAGTCCCCCG
GCCACGGGGCCTGCCACCATACCCACGCCGAAACAAGCGCTCATGAGCCC
GAAGTGGCGAGCCCGATCTTCCCCATCGGTGATGTCGGCGATATAGGCGC
CAGCAACCGCACCTGTGGCGCCGGTGATGCCGGCCACGATGCGTCCGGCG
TAGAGGCGATTAGTCCAATTTGTTAAAGACAGGATATCAGTGGTCCAGGC
TCTAGTTTTGACTCAACAATATCACCAGCTGAAGCCTATAGAGTACGAGC
CATAGATAAAATAAAAGATTTTATTTAGTCTCCAGAAAAAGGGGGGAA.

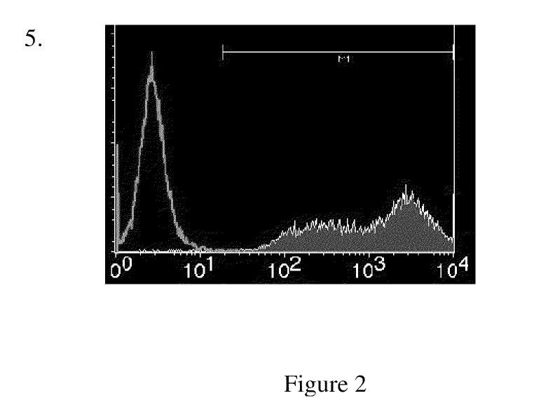

Another FLIP insert comprising loxP 5171 and loxP 2272 genes, a modified puromycin resistance cassette incorporating the foot-and-mouth-disease virus (FMDV) 2A encoding sequence at its C-terminus, fused in frame to a gene encoding GFP, and nucleic acids encoding miR30 and GFP, was similarly constructed and is outlined in FIG. 2.

The complete sequence of this construct, pPRIME-201 with puro2A-GFP insert, is as follows (upper case bases 3210-4628):

[SEQ ID NO:8]

gtcgacggatcgggagatctcccgatcccctatggtgcactctcagtaca
atctgctctgatgccgcatagttaagccagtatctgctccctgcttgtgt
gttggaggtcgctgagtagtgcgcgagcaaaatttaagctacaacaaggc
aaggcttgaccgacaattgcatgaagaatctgcttagggttaggcgtttt
gcgctgcttcgcgatgtacgggccagatatacgcgttgacattgattatt
gactagttattaatagtaatcaattacggggtcattagttcatagcccat
atatggagttccgcgttacataacttacggtaaatggcccgcctggctga
ccgcccaacgacccccgcccattgacgtcaataatgacgtatgttcccat
agtaacgccaatagggactttccattgacgtcaatgggtggagtatttac
ggtaaactgcccacttggcagtacatcaagtgtatcatatgccaagtacg
ccccctattgacgtcaatgacggtaaatggcccgcctggcattatgccca
gtacatgaccttatgggactttcctacttggcagtacatctacgtattag
tcatcgctattaccatggtgatgcggttttggcagtacatcaatgggcgt
ggatagcggtttgactcacggggatttccaagtctccaccccattgacgt
caatgggagtttgttttggcaccaaaatcaacgggactttccaaaatgtc
gtaacaactccgccccattgacgcaaatgggcggtaggcgtgtacggtgg
gaggtctatagaccagatctgagcctgggagctctctggctaactaggga
acccactgcttaagcctcaataaagcttgccttgagtgcttcaagtagtg
tgtgcccgtctgttgtgtgactctggtaactagagatccctcagaccctt
ttagtcagtgtggaaaatctctagcagtggcgcccgaacagggacttgaa
agcgaaagggaaaccagaggagctctctcgacgcaggactcggcttgctg
aagcgcgcacggcaagaggcgaggggcggcgactggtgagtacgccaaaa
attttgactagcggaggctagaaggagagagatgggtgcgagagcgtcag
tattaagcgggggagaattagatcgcgatgggaaaaaattcggttaaggc
cagggggaaagaaaaaatataaataaacatatagtatgggcaagcaggga
gctagaacgattcgcagttaatcctggcctgttagaaacatcagaaggct
gtagacaaatactgggacagctacaaccatcccttcagacaggatcagaa
gaacttagatcattatataatacagtagcaaccctctattgtgtgcatca
aaggatagagataaaagacaccaaggaagctttagacaagatagaggaag
agcaaaacaaaagtaagaccaccgcacagcaagcggccggccgcgctgat
cttcagacctggaggaggagatatgagggacaattggagaagtgaattat
ataaatataaagtagtaaaaattgaaccattaggagtagcacccaccaag
gcaaagagaagagtggtgcagagagaaaaaagagcagtgggaataggagc
tttgttccttgggttcttgggagcagcaggaagcactatgggcgcagcgt
caatgacgctgacggtacaggccagacaattattgtctggtatagtgcag
cagcagaacaatttgctgagggctattgaggcgcaacagcatctgttgca
actcacagtctggggcatcaagcagctccaggcaagaatcctggctgtgg
aaagatacctaaaggatcaacagctcctggggatttggggttgctctgga
aaactcatttgcaccactgctgtgccttggaatgctagttggagtaataa
atctctggaacagatttggaatcacacgacctggatggagtgggacagag
aaattaacaattacacaagcttaatacactccttaattgaagaatcgcaa
aaccagcaagaaaagaatgaacaagaattattggaattagataaatgggc
aagtttgtggaattggtttaacataacaaattggctgtggtatataaaat
tattcataatgatagtaggaggcttggtaggtttaagaatagtttttgct
gtactttctatagtgaatagagttaggcagggatattcaccattatcgtt
tcagacccacctcccaaccccgaggggacccgacaggcccgaaggaatag
aagaagaaggtggagagagagacagagacagatccattcgattagtgaac
ggatcggcactgcgtgcgccaattctgcagacaaatggcagtattcatcc
acaattttaaaagaaaaggggggattggggggtacagtgcaggggaaaga
atagtagacataatagcaacagacatacaaactaaagaattacaaaaaca
aattacaaaaattcaaaattttcgggtttattacagggacagcagagatc
cagtttggttagtaccgggcccgctctagacgtattaccgccatgcatta
gttattaatagtaatcaattacggggtcattagttcatagcccatatatg
gagttccgcgttacataacttacggtaaatggcccgcctggctgaccgcc
caacgacccccgcccattgacgtcaataatgacgtatgucccatagtaac
gccaatagggactttccattgacgtcaatgggtggagtatttacggtaaa
ctgcccacttggcagtacatcaagtgtatcatatgccaagtacgccccct
attgacgtcaatgacggtaaatggcccgcctggcattatgcccagtacat
gaccttatgggactttcctacttggcagtacatctacgtattagtcatcg
ctattaccatggtgatgcggttttggcagtacatcaatgggcgtggatag
cggtttgactcacggggatttccaagtctccaccccattgacgtcaatgg
gagtttgttttggcaccaaaatcaacgggactttccaaaatgtcgtaaca
actccgccccattgacgcaaatgggcggtaggcgtgtacggtgggaggtc
tatataagcagagctggtttagtgaaccgtcagatccgctagcgctaccg
gtGCCACCATGGTGGAGTACAAGCCCACGGTGCGCCTCGCCACCCGCGAC
GACGTCCCCAGGGCCGTACGCACCCTCGCCGCCGCGTTCGCCGACTACCC
CGCCACGCGCCACACCGTCGATCCGGACCGCCACATCGAGCGGGTCACCG
AGCTGCAAGAACTCTTCCTCACGCGCGTCGGGCTCGACATCGGCAAGGTG
TGGGTCGCGGACGACGGCGCCGCGGTGGCGGTCTGGACCACGCCGGAGAG
CGTCGAAGCGGGGGCGGTGTTCGCCGAGATCGGCCCGCGCATGGCCGAGT
TGAGCGGTTCCCGGCTGGCCGCGCAGCAACAGATGGAAGGCCTCCTGGCG
CCGCACCGGCCCAAGGAGCCCGCGTGGTTCCTGGCCACCGTCGGCGTCTC
GCCCGACCACCAGGGCAAGGGTCTGGGCAGCGCCGTCGTGCTCCCCGGAG
TGGAGGCGGCCGAGCGCGCCGGGGTGCCCGCCTTCCTGGAGACCTCCGCG
CCCCGCAACCTCCCCTTCTACGAGCGGCTCGGCTTCACCGTCACCGCCGA
CGTCGAGGTGCCCGAAGGACCGCGCACCTGGTGCATGACCCGCAAGCCCG
GTGCCAAACAGAAAATTGTGGCACCAGTGAAACAGACTTTGAATTTTGAC
CTTCTCAAGTTGGCGGGAGACGTCGAGTCCAACCCTGGGCCCGGCCCGGT
CGCCACCATGGTGAGCAAGGGCGAGGAGCTGTTCACCGGGGTGGTGCCCA
TCCTGGTCGAGCTGGACGGCGACGTAAACGGCCACAAGTTCAGCGTGTCC
GGCGAGGGCGACGGCGATGCCACCTACGGCAAGCTGACCCTGAAGTTCAT
CTGCACCACCGGCAAGCTGCCCGTGCCCTGGCCCACCCTCGTGACCACCC
TGACCTACGGCGTGCAGTGCTTCAGCCGCTACCCCGACCACATGAAGCAG
CACGACTTCTTCAAGTCCGCCATGCCCGAAGGCTACGTCCAGGAGCGCAC
CATCTTCTTCAAGGACGACGGCAACTACAAGACCCGCGCCGAGGTGAAGT
TCGAGGGCGACACCCTGGTGAACCGCATCGAGCTGAAGGGCATCGACTTC
AAGGAGGACGGCAACATCCTGGGGCACAAGCTGGAGTACAACTACAACAG
CCACAACGTCTATATCATGGCCGACAAGCAGAAGAACGGCATCAAGGTGA
ACTTCAAGATCCGCCACAACATCGAGGACGGCAGCGTGCAGCTCGCCGAC
CACTACCAGCAGAACACCCCCATCGGCGACGGCCCCGTGCTGCTGCCCGA
CAACCACTACCTGAGCACCCAGTCCGCCCTGAGCAAAGACCCCAACGAGA
AGCGCGATCACATGGTCCTGCTGGAGTTCGTGACCGCCGCCGGGATCACT
CTCGGCATGGACGAGCTGTACAAGTAGcggccgcaagccttgttaagtgc
tcgcttcggcagcacatatactatgtttgaatgaggcttcagtactttac
agaatcgttgcctgcacatcttggaaacacttgctgggattacttcttca
ggttaacccaacagaaggctcgagcaaccagaattcaaggggctacttta
ggagcaattatcttgtttactaaaactgaataccttgctatctctttgat
acatttttacaaagctgaattaaaatggtataaattaaatcacttttttc
aattggaagactaatgcgtttaaacacgcggcgacgcgttcgaccgaata
aaacctgtgacggaagatcacttcgcagaataaataaatcctggtgtccc
tgttgataccgggaagccctgggccaacttttggcgaaaatgagacgttg
atcggcacgtaagaggttccaactttcaccataatgaaataagatcacta
ccgggcgtattttttgagttgtcgagattttcaggagctaaggaagctaa
aatggagaaaaaaatcactggatataccaccgttgatatatcccaatggc
atcgtaaagaacattttgaggcatttcagtcagttgctcaatgtacctat
aaccagaccgttcagctggatattacggcctttttaaagaccgtaaagaa
aaataagcacaagttttatccggcctttattcacattcttgcccgcctga
tgaatgctcatccggaattacgtatggcaatgaaagacggtgagctggtg
atatgggatagtgttcacccttgttacaccgttttccatgagcaaactga
aacgttttcatcgctctggagtgaataccacgacgatttccggcagtttc
tacacatatattcgcaagatgtggcgtgttacggtgaaaacctggcctat
ttccctaaagggtttattgagaatatgtttttcgtctcagccaatccctg
ggtgagtttcaccagttttgatttaaacgtggccaatatggacaacttct
tcgcccccgttttcaccatgggcaaatattatacgcaaggcgacaaggtg
ctgatgccgctggcgattcaggttcatcatgccgtttgtgatggcttcca
tgtcggcagaatgcttaatgaattacaacagtactgcgatgagtggcagg
gcggggcgtaatttttttaaggcagttattggtgcccttaaacgcctggt
tgctacgcctgaataagtgataataagcggatgaatggcagaaattcgga
tctcgaccgcgtttgggcggtggctccctgccacgcggctccgaacagaa
gctgatctccgaagaggatctgacatgtgtttaaacctcgacttaattaa
gtcgagggtcgacggtatcgataagctcgcttcacgagatcatgtttaag
ggttccggttccactaggtacaattcgatatcaagcttatcgataatcaa
cctctggattacaaaatttgtgaaagattgactggtattcttaactatgt
tgctccttttacgctatgtggatacgctgctttaatgcctttgtatcatg
ctattgcttcccgtatggctttcattttctcctccttgtataaatcctgg
ttgctgtctctttatgaggagttgtggcccgttgtcaggcaacgtggcgt
ggtgtgcactgtgtttgctgacgcaacccccactggttggggcattgcca
ccacctgtcagctcctttccgggactttcgctttccccctccctattgcc
acggcggaactcatcgccgcctgccttgcccgctgctggacaggggctcg
gctgttgggcactgacaattccgtggtigttgtcggggaaatcatcgtcc
tttccttggctgctcgcctgtgttgccacctggauctgcgcgggacgtcc
ttctgctacgtcccttcggccctcaatccagcggaccttccttcccgcgg
cctgctgccggctctgcggcctcttccgcgtcttcgccttcgccctcaga
cgagtcggatctccctttgggccgcctccccgcatcgataccgtcgacct
cgatcgagacctagaaaaacatggagcaatcacaagtagcaatacagcag
ctaccaatgctgattgtgcctggctagaagcacaagaggaggaggaggtg
ggttttccagtcacacctcaggtacctttaagaccaatgacttacaaggc
agctgtagatcttagccactttttaaaagaaaaggggggactggaagggc
taattcactcccaacgaagacaagatatccttgatctgtggatctaccac
acacaaggctacttccctgattggcagaactacacaccagggccagggat
cagatatccactgacctttggatggtgctacaagctagtaccagttgagc
aagagaaggtagaagaagccaatgaaggagagaacacccgcttgttacac
cctgtgagcctgcatgggatggatgacccggagagagaagtattagagtg
gaggtttgacagccgcctagcatttcatcacatggcccgagagctgcatc
cggactgtactgggtctctctggttagaccagatctgagcctgggagctc
tctggctaactagggaacccactgcttaagcctcaataaagcttgccttg
agtgcttcaagtagtgtgtgcccgtctgttgtgtgactctggtaactaga
gatccctcagacccttttagtcagtgtggaaaatctctagcagcatgtga
gcaaaaggccagcaaaaggccaggaaccgtaaaaaggccgcgttgctggc
gtttttccataggctccgcccccctgacgagcatcacaaaaatcgacgct
caagtcagaggtggcgaaacccgacaggactataaagataccaggcgttt
ccccctggaagctccctcgtgcgctctcctgttccgaccctgccgcttac
cggatacctgtccgcctttctcccttcgggaagcgtggcgctttctcata
gctcacgctgtaggtatctcagttcggtgtaggtcgttcgctccaagctg
ggctgtgtgcacgaaccccccgttcagcccgaccgctgcgccttatccgg
taactatcgtcttgagtccaacccggtaagacacgacttatcgccactgg
cagcagccactggtaacaggattagcagagcgaggtatgtaggcggtgct
acagagttcttgaagtggtggcctaactacggctacactagaagaacagt
atttggtatctgcgctctgctgaagccagttaccttcggaaaaagagttg
gtagctcttgatccggcaaacaaaccaccgctggtagcggtggttttttt
gtttgcaagcagcagattacgcgcagaaaaaaaggatctcaagaagatcc
tttgatcttttctacggggtctgacgctcagtggaacgaaaactcacgtt
aagggattttggtcatgagattatcaaaaaggatcttcacctagatcctt
ttaaattaaaaatgaagttttaaatcaatctaaagtatatatgagtaaac
ttggtctgacagttaccaatgcttaatcagtgaggcacctatctcagcga
tctgtctatttcgttcatccatagttgcctgactccccgtcgtgtagata
actacgatacgggagggcttaccatctggccccagtgctgcaatgatacc
gcgagacccacgctcaccggctccagatttatcagcaataaaccagccag
ccggaagggccgagcgcagaagtggtcctgcaactttatccgcctccatc
cagtctattaattgttgccgggaagctagagtaagtagttcgccagttaa
tagtttgcgcaacgttgttgccattgctacaggcatcgtggtgtcacgct
cgtcgtttggtatggcttcattcagctccggttcccaacgatcaaggcga
gttacatgatcccccatgttgtgcaaaaaagcggttagctccttcggtcc
tccgatcgttgtcagaagtaagttggccgcagtgttatcactcatggtta
tggcagcactgcataattctcttactgtcatgccatccgtaagatgcttt
tctgtgactggtgagtactcaaccaagtcattctgagaatagtgtatgcg
gcgaccgagttgctcttgcccggcgtcaatacgggataataccgcgccac
atagcagaactttaaaagtgctcatcattggaaaacgttcttcggggcga
aaactctcaaggatcttaccgctgttgagatccagttcgatgtaacccac
tcgtgcacccaactgatcttcagcatcttttactttcaccagcgtttctg
ggtgagcaaaaacaggaaggcaaaatgccgcaaaaaagggaataagggcg
acacggaaatgttgaatactcatactcttcctttttcaatattattgaag
catttatcagggttattgtctcatgagcggatacatatttgaatgtattt
agaaaaataaacaaataggggttccgcgcacatttccccgaaaagtgcca
cctgac.

MSCV-Cre was obtained from the laboratory of Luk Van Parijs. This vector to express Cre recombinase was a variant of pMIG in which the GFP reporter was replaced by the human surface molecule CD8 that has a deletion fo the cytoplasmic tail. Cre was cloned into this MICD8 vector by digestion with BglII and EcoRI and ligation.

To generate MCSV-Cre, pMIG (Grez et al., Proc. Natl. Acad. Sci. USA 87:9202-9206, 1990) was modified, as follows: the vector was digested with BglII and EcoRI, the Cre insert replaced ires-GFP, followed by insert fill in and ligation.

### Reagents

Anti p53 antibody was provided by Andrea Ventura. Doxorubicin and doxycycline were obtained from Sigma.

### Verification of Reporter Expression:

Lewis Lung carcinoma cells (LL2) were transducted with pFLIP and probed for Thy 1.1 expression by FACS analysis following puromycin selection. Selected cells were also probed for GFP expression, prior to and following infection with MCSV-Cre.

Infectious viral particles were produced through standard lab methods. 293FT cells were transiently transfected with retroviral gag/pol and VSVg envelopes plasmids along with the viral vector. The supernatants were harvested at 48 hours and used to infect target cells in the presence of 4 ug/ml polybrene.

### In Vitro Knockdown Studies

Primary mouse embryo fibroblasts (MEFs) were infected with MSCV retrovirus expressing pFLIP encoding RNAi against the tumor suppressor p53, or luciferase. Cells were selected with puromycin and infected with MSCV-Cre, treated for 5 hours with doxorubicin, and p53 expression was assayed by Western blotting. GAPDH served as a protein loading control.

### Example 1

### Construction of Stable, Cre-lox Based Knockdown Constructs

The pFLIP construct is schematically depicted in FIG. 1. The construct may be expressed by a constitutive, tissue-specific, or inducible promoter. The mRNA expresses puromycin resistance and the surface marker Thy1.1 or GFP. The puromycin-Thy1.1, or -GFP construct, respectively, is translated as a fusion protein but generates two distinct polypeptides by virtue of the 2A peptide at the C-terminus of the puromycin resistance or GFP cassette, which results in the translation of two distinct polypeptide species from a single cistron.

As depicted in FIG. 1, the green fluorescent protein (GFP) and a miR30 microRNA-based, RNAi construct are present in the anti-sense orientation in the 3′ untranslated region of the mRNA. Upon addition of Cre recombinase, the puro-Thy1.1 cassette is deleted and the GFP-microRNA construct is reversed to the sense orientation, allowing expression of GFP and RNAi.

In this embodiment, the vector expresses two markers a drug selection and a surface marker. When Cre is introduced, the markers are deleted and expression of GFP and RNAi is induced. GFP and the RNAi are in antisense orientation until Cre-recombinase is active, at which point there is a “flip” to the sense orientation.

### Example 2

### pFLIP Constructs are Stably Expressed Following Cre-Mediated Recombination

In order to determine whether the construct design in Example 1 results in conditional knockdown of specific gene expression, FACS analysis of marker expression was conducted on cells transduced with the pFLIP constructs described in an “uninduced” (no Cre recombinase supplied) and “induced” state (following Cre supply) (FIG. 2).

Lewis Lung carcinoma cell line (LL2) transduced with pFLIP-MSCV and selected with puromycin expressed the surface marker Thy1.1. (panel 2), in contrast to uninfected negative controls. These cells did not express GFP in the “uninduced” state, i.e. prior to exposure to MSCV-Cre (panel 3), while GFP expression was readily evident in cells infected with the Cre-expressing retrovirus (Panel 4). GFP expression was readily evident in MEF cells transduced with pFLIP-MSCV expressing the puromycin-GFP construct described in Example 1, when subjected to puromycin selection (FIG. 2, panel 5)

### Example 3

### Conditional Gene Knockdown with pFLIP Constructs

Primary mouse embryo fibroblasts (MEFs) expressing pFLIP encoding an RNAi against the tumor suppressor p53 conditionally knocked down p53 expression, when infected with MSCV retrovirus (FIG. 3). Cells treated with doxorubicin, which causes DNA damage and induces p53 expression, selected with puromycin demonstrated p53 expression, in “uninduced” conditions, however, when infected with a retrovirus expressing Cre recombinase, showed p53 knockdown, as seen by reduced p53 staining in Western Blots (lane 4).

Thus efficient gene knockdown was accomplished with the pFLIP constructs of this invention, specifically under conditions of Cre-mediated induction.

Knockdown was accomplished by recombinantly expressed miRNA. It is thought that while the miRNA sequence is in the antisense orientation, it can still fold to form a stem-loop structure, which is thought to be necessary for miRNA activity, thus background knockdown of gene expression might have been predicted with the constructs of this invention, yet surprisingly, this was not the case. The microRNA in antisense orientation was not processed to generate effective RNAi intermediates, nor did the antisense disrupt marker gene translation or virus production, indicating that the constructs provide for specific, controlled regulation of gene expression.

The foregoing has been a description of certain non-limiting preferred embodiments of the invention. Those of ordinary skill in the art will appreciate that various changes and modifications to this description may be made without departing from the spirit or scope of the present invention, as defined in the following claims.

What is claimed is:

1. A vector comprising:
i. a first pair of loxP sequences, inverted in orientation, with respect to each other;
ii. a first nucleic acid encoding a first selectable marker in sense orientation, wherein said nucleic acid is positioned between said first pair of loxP sequences;
iii. a second nucleic acid encoding a second selectable marker, fused in frame to an miRNA sequence of interest in antisense orientation, said second nucleic acid is positioned between said first pair of loxP sequences, and said second nucleic acid is 3′ with regard to said first nucleic acid;
iv. a second pair of loxP sequences, inverted in orientation, with respect to each other, wherein said first loxP sequenced of said second pair is positioned between said first and said second nucleic acid, and said second loxP sequence in said second pair is positioned 3′ with respect to said first pair of loxP sequences, and said second pair of loxP sequences differs from that of said first pair of loxP sequences.

2. The vector of claim 1, wherein said first pair of loxP sequences comprises the wildtype sequence.

3. The vector of claim 1, wherein said second pair of loxP sequences comprises a mutated loxP.

4. The vector of claim 1, wherein said first pair of loxP sequences comprises the loxP 5171 sequence.

5. The vector of claim 1, wherein said second pair of loxP sequences comprises the loxP 2272 sequence.

6. The vector of claim 1, wherein said first nucleic acid encodes two selectable markers fused in-frame with respect to each other.

7. The vector of claim 6, wherein said two selectable markers comprise a first antibiotic resistance cassette fused in frame to a sequence encoding a cell surface marker.

8. The vector of claim 6, wherein said first of said two markers comprises a c-terminal sequence encoding a Foot-and-mouth-disease virus (FMDV) 2A peptide.

9. The vector of claim 8, wherein said two selectable markers localize to different cellular compartments, when expressed.

10. The vector of claim 1, wherein said vector comprises a promoter operatively linked to said first nucleic acid.

11. The vector of claim 10, wherein said promoter is tissue specific.

12. The vector of claim 10, wherein said promoter is inducible.

13. The vector of claim 1, wherein said miRNA agent is an shRNA.

14. The vector of claim 1, wherein said miRNA specifically inactivates p53 gene expression.

15. The vector of claim 1, wherein said miRNA sequence corresponds to, or is homologous to SEQ ID NO: 5.

16. The vector of claim 15, wherein said vector comprise a nucleotide sequence corresponding to, or homolgous to SEQ ID NO: 6.

17. The vector of claim 15, wherein said vector has a nucleotide sequence corresponding to SEQ ID NO: 7.

18. The vector of claim 1, wherein the backbone of said vector is derived from a retrovirus.

19. A composition or cell comprising the vector of claim 1.

20. A method of producing an animal genetically inactivated for a coding sequence, the method comprising:
a. contacting an embryonic stem cell with the vector of claim 1;
b. injecting the embryonic stem cell in (a) to a blastocyst of said animal; and
c. obtaining an animal in (b) expressing said vector
whereby, following Cre-mediated recombination in said animal, said miRNA agent is expressed and reduces expression of said coding sequence, thereby being a method of producing an animal genetically inactivated for a coding sequence.

21. The method of claim 20, wherein said second selectable marker is expressed in a plurality of cells of said animal following Cre-mediated recombination.

22. The method of claim 20, wherein expression of said first selectable marker is lost in a plurality of cells of said animal, following Cre-mediated recombination.

23. A method of conditionally reducing expression of a coding sequence in a target cell, said method comprising contacting said target cell with a vector comprising:
i. a first pair of loxP sequences, inverted in orientation, with respect to each other;
ii. a first nucleic acid encoding a first selectable marker in sense orientation, wherein said nucleic acid is positioned between said first pair of loxP sequences;
iii. a second nucleic acid encoding a second selectable marker, fused in frame to an miRNA sequence of interest in antisense orientation, said second nucleic acid is positioned between said first pair of loxP sequences, and said second nucleic acid is 3′ with regard to said first nucleic acid;
iv. a second pair of loxP sequences, inverted in orientation, with respect to each other, wherein said first loxP sequenced of said second pair is positioned between said first and said second nucleic acid, and said second loxP sequence in said second pair is positioned 3′ with respect to said first pair of loxP sequences, and said second pair of loxP sequences differs from that of said first pair of loxP sequences.

24. The method according to claim 23, wherein said cell is engineered to express a Cre recombinase.

25. The method according to claim 23, wherein said cell endogenously expresses a Cre recombinase.

26. The method according to claim 23, wherein said target cell is contacted with said vector in vivo, in vitro or ex-vivo.

27. The method according to claim 26, wherein said cell is in vivo, and said Cre recombinase is expressed at specific times during development.

28. The method according to claim 23, wherein said first pair of loxP sequences comprises the wildtype sequence.

29. The method according to claim 23, wherein said second pair of loxP sequences comprises a mutated loxP.

30. The method according to claim 23, wherein said first pair of loxP sequences comprises the loxP 5171 sequence.

31. The method according to claim 23, wherein said second pair of loxP sequences comprises the loxP 2272 sequence.

32. The method according to claim 23, wherein said first nucleic acid encodes two selectable markers fused in-frame with respect to each other.

33. The method according to claim 32, wherein said two selectable markers comprise a first antibiotic resistance cassette fused in frame to a sequence encoding a cell surface marker.

34. The method according to claim 32, wherein said first of said two markers comprises a c-terminal sequence encoding a Foot-and-mouth-disease virus (FMDV) 2A peptide.

35. The method according to claim 34, wherein said two selectable markers localize to different cellular compartments, when expressed.

36. The method according to claim 23, wherein said vector comprises a promoter operatively linked to said first nucleic acid.

37. The method according to claim 36, wherein said promoter is tissue specific.

38. The method according to claim 36, wherein said promoter is inducible.

39. The method according to claim 23, wherein said miRNA agent is an shRNA.

40. The method according to claim 23, wherein said miRNA specifically inactivates p53 gene expression.

41. The method according to claim 23, wherein said miRNA sequence corresponds to, or is homologous to SEQ ID NO: 5.

42. The method according to claim 41, wherein said vector comprise a nucleotide sequence corresponding to, or homolgous to SEQ ID NO: 6.

43. The method according to claim 41, wherein said vector has a nucleotide sequence corresponding to SEQ ID NO: 7.

44. A non-human animal with reduced expression of a coding sequence, wherein said reduced expression is produced according to the method of claim 23.

45. A mammalian cell with reduced expression of a coding sequence, wherein said reduced expression is produced in said cell according to the method of claim 23.

46. A method of conditionally expressing a coding sequence in a target cell, the method comprising contacting said target cell with a vector comprising:
i. a first pair of loxP sequences, inverted in orientation, with respect to each other;
ii. a first nucleic acid encoding a first selectable marker in sense orientation, fused in frame to an miRNA sequence of interest, wherein said nucleic acid is positioned between said first pair of loxP sequences;
iii. a second nucleic acid encoding a second selectable marker, in antisense orientation, said second nucleic acid is positioned between said first pair of loxP sequences, and said second nucleic acid is 3′ with regard to said first nucleic acid;
iv. a second pair of loxP sequences, inverted in orientation, with respect to each other, wherein said first loxP sequenced of said second pair is positioned between said first and said second nucleic acid, and said second loxP sequence in said second pair is positioned 3′ with respect to said first pair of loxP sequences, and said second pair of loxP sequences differs from that of said first pair of loxP sequences.
wherein said cell expresses said miRNA agent, thereby reducing expression of said coding sequence and whereby, following Cre-mediated recombination in said target cell, said miRNA agent is no longer expressed, thereby being a method of conditionally expressing a coding sequence in a target cell.

47. The method according to claim 46, wherein said cell is engineered to express a Cre recombinase.

48. The method according to claim 46, wherein said cell endogenously expresses a Cre recombinase.

49. The method according to claim 46, wherein said target cell is contacted with said vector in vivo, in vitro or ex-vivo.

50. The method according to claim 49, wherein said cell is in vivo, and said Cre recombinase is expressed at specific times during development.

51. The method according to claim 46, wherein said first pair of loxP sequences comprises the wildtype sequence.

52. The method according to claim 46, wherein said second pair of loxP sequences comprises a mutated loxP.

53. The method according to claim 46, wherein said first pair of loxP sequences comprises the loxP 5171 sequence.

54. The method according to claim 46, wherein said second pair of loxP sequences comprises the loxP 2272 sequence.

55. The method according to claim 46, wherein said second nucleic acid encodes two selectable markers fused in-frame with respect to each other.

56. The method according to claim 55, wherein said two selectable markers comprise a first antibiotic resistance cassette fused in frame to a sequence encoding a cell surface marker.

57. The method according to claim 55, wherein said first of said two markers comprises a c-terminal sequence encoding a Foot-and-mouth-disease virus (FMDV) 2A peptide.

58. The method according to claim 57, wherein said two selectable markers localize to different cellular compartments, when expressed.

59. The method according to claim 46, wherein said vector comprises a promoter operatively linked to said first nucleic acid.

60. The method according to claim 59, wherein said promoter is tissue specific.

61. The method according to claim 59, wherein said promoter is inducible.

62. The method according to claim 46, wherein said miRNA agent is an shRNA.

63. The method according to claim 46, wherein said miRNA specifically inactivates p53 gene expression.

64. The method according to claim 46, wherein said miRNA sequence corresponds to, or is homologous to SEQ ID NO: 5.

65. The method according to claim 64, wherein said vector comprise a nucleotide sequence corresponding to, or homolgous to SEQ ID NO: 6.

66. The method according to claim 65, wherein said vector has a nucleotide sequence corresponding to SEQ ID NO: 7.

67. A non-human animal with reactivated expression of a coding sequence, wherein said reactivated expression is produced according to the method of claim 46.

68. A mammalian cell with reactivated expression of a coding sequence, wherein said reactivated expression is produced according to the method of claim 46.

69. A vector comprising:
i. a first pair of loxP sequences, inverted in orientation, with respect to each other;
ii. a first nucleic acid encoding a first selectable marker in sense orientation, fused in frame to an miRNA sequence of interest, wherein said nucleic acid is positioned between said first pair of loxP sequences;
iii. a second nucleic acid encoding a second selectable marker, in antisense orientation, said second nucleic acid is positioned between said first pair of loxP sequences, and said second nucleic acid is 3′ with regard to said first nucleic acid; and
iv. a second pair of loxP sequences, inverted in orientation, with respect to each other, wherein said first loxP sequenced of said second pair is positioned between said first and said second nucleic acid, and said second loxP sequence in said second pair is positioned 3′ with respect to said first pair of loxP sequences, and said second pair of loxP sequences differs from that of said first pair of loxP sequences.

70. The vector claim 69, wherein said first pair of loxP sequences comprises the wildtype sequence.

71. The vector claim 69, wherein said second pair of loxP sequences comprises a mutated loxP.

72. The vector claim 69, wherein said first pair of loxP sequences comprises the loxP 5171 sequence.

73. The vector claim 69, wherein said second pair of loxP sequences comprises the loxP 2272 sequence.

74. The vector claim 69, wherein said second nucleic acid encodes two selectable markers fused in-frame with respect to each other.

75. The vector claim 74, wherein said two selectable markers comprise a first antibiotic resistance cassette fused in frame to a sequence encoding a cell surface marker.

76. The vector claim 74, wherein said first of said two markers comprises a c-terminal sequence encoding a Foot-and-mouth-disease virus (FMDV) 2A peptide.

77. The vector claim 76, wherein said two selectable markers localize to different cellular compartments, when expressed.

78. The vector claim 69, wherein said vector comprises a promoter operatively linked to said first nucleic acid.

79. The vector claim 78, said promoter is tissue specific.

80. The vector claim 78, wherein said promoter is inducible.

81. The vector claim 69, wherein said miRNA agent is an shRNA.

82. The vector claim 69, wherein said miRNA specifically inactivates p53 gene expression.

83. The vector claim 69, wherein said miRNA sequence corresponds to, or is homologous to SEQ ID NO: 5.

84. The vector claim 83, wherein said vector comprise a nucleotide sequence corresponding to, or homolgous to SEQ ID NO: 6.

85. The vector claim 83, wherein said vector has a nucleotide sequence corresponding to SEQ ID NO: 7.

86. A method of producing an animal genetically reactivated for a coding sequence, the method comprising:
a. contacting a single cell embryo of said animal with the vector of claim 69; and
b. obtaining an animal expressing said vector
whereby following Cre-mediated recombination in said animal, said miRNA agent is no longer expressed and said coding sequence is expressed, thereby being a method of producing an animal genetically reactivated for a coding sequence.
